SEQUENCE LISTING (110 sequences)

SEQ ID NO: 1 - protein, 13 aa, Artificial sequence
  Synthetic peptide
  MISC_FEATURE (1)..(1): Xaa = absent or Gly
  MISC_FEATURE (2)..(2): Xaa = Tyr, D-Tyr, NMe-Tyr, or absent
  MISC_FEATURE (3)..(3): Xaa = Val, Gly, Ala, Aib, or absent
  MISC_FEATURE (4)..(4): Xaa = Met, Nle, Glu, Ser, Asp, homoGlu, or Thr
  MISC_FEATURE (5)..(5): Xaa = Gly, D-Ala, D-Val, D-Nle, NMe-D-Ala, D-Pro, Ala, Aib, D-Abu, D-Phe, Glu, or absent
  MISC_FEATURE (6)..(6): Xaa = His, Pro, Gln, Cit, NMe-His, NMe-Arg, NMe-Lys, NMe-Ala
  MISC_FEATURE (7)..(7): Xaa = Phe, D-Phe, Nal(2′), Trp, D-Trp, Tic, Hph, Bip, D-Bip, aMe-Phe, D-Phe(4-NH-Ac), Phe(4-F), Phe(4tBu), Phe(4-Br), Phe(4-I), Phe(4-Cl), D-Phe(4-Br), D-Phe(4-I), NMe-Phe, or D-Phe(4-Cl)
  MISC_FEATURE (8)..(8): Xaa = Arg, D-Arg, Lys, Orn, Cit, or Nle
  MISC_FEATURE (9)..(9): Xaa = aMe-D-Trp, D-Trp, Trp, D-Nal(2′), D-Phe, D-Tic, Tic, D-Phe, or D-Ala
  MISC_FEATURE (10)..(10): Xaa = Asp, Ala, Phe, D-Phe, Nle, Lys, Gly, Orn, or absent
  MISC_FEATURE (11)..(11): Xaa = Arg, D-Arg, Lys, Ala, or absent
  MISC_FEATURE (12)..(12): Xaa = Phe, D-Phe, Pro, Gly, Ala, or absent
  MISC_FEATURE (13)..(13): Xaa = Gly, Lys, Val, or absent
XXXXXXXXXXXXX

SEQ ID NO: 2 - protein, 12 aa, Artificial sequence
  Synthetic peptide
  MISC_FEATURE (8)..(8): D-Trp
YVMGHFRWDRFG

SEQ ID NO: 3 - protein, 12 aa, Artificial sequence
  Synthetic peptide
  MISC_FEATURE (3)..(3): Xaa = Nle
  MISC_FEATURE (4)..(4): D-Ala
  MISC_FEATURE (8)..(8): D-Trp
YVXAHFRWDRFG

SEQ ID NO: 4 - protein, 9 aa, Artificial sequence
  Synthetic peptide
  MISC_FEATURE (3)..(3): Xaa = Nle
  MISC_FEATURE (4)..(4): D-Ala
  MISC_FEATURE (8)..(8): D-Trp
YVXAHFRWD

SEQ ID NO: 5 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRWDRFG

SEQ ID NO: 6 - protein, 11 aa, Artificial sequence
  Synthetic peptide
  MISC_FEATURE (2)..(2): Xaa = Nle
  MISC_FEATURE (3)..(3): D-Ala
  MISC_FEATURE (7)..(7): D-Trp
VXAHFRWDRFG

SEQ ID NO: 7 - protein, 12 aa, Artificial sequence
  Synthetic peptide
  MISC_FEATURE (1)..(1): D-Tyr
  MISC_FEATURE (3)..(3): Xaa = Nle
  MISC_FEATURE (4)..(4): D-Ala
  MISC_FEATURE (8)..(8): D-Trp
YVXAHFRWDRFG

SEQ ID NO: 8 - protein, 12 aa, Artificial sequence
  Synthetic peptide
  MISC_FEATURE (3)..(3): Xaa = Nle
  MISC_FEATURE (8)..(8): D-Trp
YVXAHFRWDRFG

SEQ ID NO: 9 - protein, 9 aa, Artificial sequence
  Synthetic peptide
  MISC_FEATURE (3)..(3): Xaa = Nle
  MISC_FEATURE (8)..(8): D-Trp
YVXAHFRWD

SEQ ID NO: 10 - protein, 12 aa, Artificial sequence
  Synthetic peptide
  MISC_FEATURE (3)..(3): Xaa = Nle
  MISC_FEATURE (4)..(4): Xaa = D-alpha-Aminobutyric acid
  MISC_FEATURE (8)..(8): D-Trp
YVXXHFRWDRFG

SEQ ID NO: 11 - protein, 9 aa, Artificial sequence
  Synthetic peptide
  MISC_FEATURE (3)..(3): Xaa = Nle
  MISC_FEATURE (4)..(4): Xaa = D-alpha-Aminobutyric acid
  MISC_FEATURE (8)..(8): D-Trp
YVXXHFRWD

SEQ ID NO: 12 - protein, 9 aa, Artificial sequence
  Synthetic peptide
  MISC_FEATURE (3)..(3): Xaa = Nle
  MISC_FEATURE (4)..(4): D-Phe
  MISC_FEATURE (8)..(8): D-Trp
YVXFHFRWD

SEQ ID NO: 13 - protein, 12 aa, Artificial sequence
  Synthetic peptide
  MISC_FEATURE (3)..(3): Xaa = Nle
  MISC_FEATURE (4)..(4): Xaa = 2-Aminoisobutyric acid
  MISC_FEATURE (8)..(8): D-Trp
YVXXHFRWDRFG

SEQ ID NO: 14 - protein, 9 aa, Artificial sequence
  Synthetic peptide
  MISC_FEATURE (3)..(3): Xaa = Nle
  MISC_FEATURE (4)..(4): Xaa = 2-Aminoisobutyric acid
  MISC_FEATURE (8)..(8): D-Trp
YVXXHFRWD

SEQ ID NO: 15 - protein, 9 aa, Artificial sequence
  Synthetic peptide
  MISC_FEATURE (3)..(3): Xaa = Nle
  MISC_FEATURE (4)..(4): D-Ala
  MISC_FEATURE (8)..(8): D-Trp
YVXAHFRWD

SEQ ID NO: 16 - protein, 9 aa, Artificial sequence
  Synthetic peptide
  MISC_FEATURE (3)..(3): Xaa = Nle
  MISC_FEATURE (4)..(4): D-Ala
  MISC_FEATURE (8)..(8): D-Trp
YVXAHFRWD

SEQ ID NO: 17 - protein, 12 aa, Artificial sequence
  Synthetic peptide
  MISC_FEATURE (3)..(3): Xaa = Nle
  MISC_FEATURE (4)..(4): D-Ala
  MISC_FEATURE (8)..(8): D-Trp
  MISC_FEATURE (10)..(10): D-Arg
YVXAHFRWDRFG

SEQ ID NO: 18 - protein, 12 aa, Artificial sequence
  Synthetic peptide
  MISC_FEATURE (3)..(3): Xaa = Nle
  MISC_FEATURE (4)..(4): D-Ala
  MISC_FEATURE (8)..(8): D-Trp
  MISC_FEATURE (11)..(11): D-Phe
YVXAHFRWDRFG

SEQ ID NO: 19 - protein, 12 aa, Artificial sequence
  Synthetic peptide
  MISC_FEATURE (8)..(8): D-Trp
YVEGHFRWDRFG

SEQ ID NO: 20 - protein, 12 aa, Artificial sequence
  Synthetic peptide
  MISC_FEATURE (3)..(3): Xaa = Nle
  MISC_FEATURE (4)..(4): D-Ala
  MISC_FEATURE (6)..(6): Phe(4-Br)
  MISC_FEATURE (8)..(8): D-Trp
YVXAHFRWDRFG

SEQ ID NO: 21 - protein, 12 aa, Artificial sequence
  Synthetic peptide
  MISC_FEATURE (3)..(3): Xaa = Nle
  MISC_FEATURE (4)..(4): D-Ala
  MISC_FEATURE (6)..(6): Phe(4-Cl)
  MISC_FEATURE (8)..(8): D-Trp
YVXAHFRWDRFG

SEQ ID NO: 22 - protein, 12 aa, Artificial sequence
  Synthetic peptide
  MISC_FEATURE (3)..(3): Xaa = Nle
  MISC_FEATURE (4)..(4): D-Ala
  MISC_FEATURE (6)..(6): Phe(4-I)
  MISC_FEATURE (8)..(8): D-Trp
YVXAHFRWDRFG

SEQ ID NO: 23 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
  MISC_FEATURE (9)..(9): D-Phe
XAHFRWDRFG

SEQ ID NO: 24 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
  MISC_FEATURE (8)..(8): D-Arg
XAHFRWDRFG

SEQ ID NO: 25 - protein, 9 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRWDRF

SEQ ID NO: 26 - protein, 9 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
  MISC_FEATURE (9)..(9): D-Phe
XAHFRWDRF

SEQ ID NO: 27 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
  MISC_FEATURE (9)..(9): D-Phe
XAHFRWDRFK

SEQ ID NO: 28 - protein, 10 aa, Artificial sequence
  Synthetic peptide
  MISC_FEATURE (1)..(1): Xaa = Nle
  MISC_FEATURE (2)..(2): D-Ala
  MISC_FEATURE (4)..(4): Phe(4-I)
  MISC_FEATURE (6)..(6): D-Trp
XAHFRWDRFG

SEQ ID NO: 29 - protein, 9 aa, Artificial sequence
  Synthetic peptide
  MISC_FEATURE (1)..(1): Xaa = Nle
  MISC_FEATURE (2)..(2): D-Ala
  MISC_FEATURE (4)..(4): Phe(4-I)
  MISC_FEATURE (6)..(6): D-Trp
XAHFRWDRF

SEQ ID NO: 30 - protein, 8 aa, Artificial sequence
  Synthetic peptide
  MISC_FEATURE (1)..(1): Xaa = Nle
  MISC_FEATURE (2)..(2): D-Ala
  MISC_FEATURE (4)..(4): Phe(4-I)
  MISC_FEATURE (6)..(6): D-Trp
XAHFRWDR

SEQ ID NO: 31 - protein, 10 aa, Artificial sequence
  Synthetic peptide
  MISC_FEATURE (1)..(1): Xaa = Nle
  MISC_FEATURE (2)..(2): Xaa = 2-Aminoisobutyric acid
  MISC_FEATURE (6)..(6): D-Trp
XXHFRWDRFG

SEQ ID NO: 32 - protein, 10 aa, Artificial sequence
  Synthetic peptide
  MISC_FEATURE (1)..(1): Xaa = Nle
  MISC_FEATURE (2)..(2): Xaa = D-alpha-Aminobutyric acid
  MISC_FEATURE (6)..(6): D-Trp
XXHFRWDRFG

SEQ ID NO: 33 - protein, 8 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRWDR

SEQ ID NO: 34 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRWGKPV

SEQ ID NO: 35 - protein, 10 aa, Artificial sequence
  Synthetic peptide
  MISC_FEATURE (1)..(1): Xaa = Nle
  MISC_FEATURE (2)..(2): D-Ala
  MISC_FEATURE (4)..(4): Phe(4-Br)
  MISC_FEATURE (6)..(6): D-Trp
XAHFRWDRFG

SEQ ID NO: 36 - protein, 10 aa, Artificial sequence
  Synthetic peptide
  MISC_FEATURE (1)..(1): Xaa = Nle
  MISC_FEATURE (2)..(2): D-Ala
  MISC_FEATURE (4)..(4): Phe(4-Cl)
  MISC_FEATURE (6)..(6): D-Trp
XAHFRWDRFG

SEQ ID NO: 37 - protein, 10 aa, Artificial sequence
  Synthetic peptide
  MISC_FEATURE (1)..(1): Xaa = Nle
  MISC_FEATURE (2)..(2): D-Ala
  MISC_FEATURE (4)..(4): Xaa = N-alpha-Fmoc-beta-(4-biphenyl)-L-alanine
  MISC_FEATURE (6)..(6): D-Trp
XAHXRWDRFG

SEQ ID NO: 38 - protein, 9 aa, Artificial sequence
  Synthetic peptide
  MISC_FEATURE (1)..(1): D-Tyr
  MISC_FEATURE (3)..(3): Xaa = Nle
  MISC_FEATURE (4)..(4): D-Ala
  MISC_FEATURE (6)..(6): Phe(4-I)
  MISC_FEATURE (8)..(8): D-Trp
YVXAHFRWD

SEQ ID NO: 39 - protein, 11 aa, Artificial sequence
  Synthetic peptide
  MISC_FEATURE (2)..(2): Xaa = Nle
  MISC_FEATURE (3)..(3): D-Ala
  MISC_FEATURE (7)..(7): D-Trp
GXAHFRWDRFG

SEQ ID NO: 40 - protein, 12 aa, Artificial sequence
  Synthetic peptide
  MISC_FEATURE (3)..(3): Xaa = Nle
  MISC_FEATURE (4)..(4): D-Ala
  MISC_FEATURE (8)..(8): D-Trp
GVXAHFRWDRFG

SEQ ID NO: 41 - protein, 13 aa, Artificial sequence
  Synthetic peptide
  MISC_FEATURE (2)..(2): D-Tyr
  MISC_FEATURE (4)..(4): Xaa = Nle
  MISC_FEATURE (5)..(5): D-Ala
  MISC_FEATURE (9)..(9): D-Trp
  MISC_FEATURE (11)..(11): D-Arg
GYVXAHFRWDRFG

SEQ ID NO: 42 - protein, 11 aa, Artificial sequence
  Synthetic peptide
  MISC_FEATURE (2)..(2): Xaa = Nle
  MISC_FEATURE (3)..(3): D-Ala
  MISC_FEATURE (7)..(7): D-Trp
GXAHFRWDRFG

SEQ ID NO: 43 - protein, 12 aa, Artificial sequence
  Synthetic peptide
  MISC_FEATURE (3)..(3): Xaa = Nle
  MISC_FEATURE (4)..(4): D-Ala
  MISC_FEATURE (8)..(8): D-Trp
GVXAHFRWDRFG

SEQ ID NO: 44 - protein, 13 aa, Artificial sequence
  Synthetic peptide
  MISC_FEATURE (2)..(2): D-Tyr
  MISC_FEATURE (4)..(4): Xaa = Nle
  MISC_FEATURE (5)..(5): D-Ala
  MISC_FEATURE (9)..(9): D-Trp
GYVXAHFRWDRFG

SEQ ID NO: 45 - protein, 11 aa, Artificial sequence
  Synthetic peptide
  MISC_FEATURE (2)..(2): Xaa = Nle
  MISC_FEATURE (3)..(3): D-Ala
  MISC_FEATURE (7)..(7): D-Trp
  MISC_FEATURE (10)..(10): D-Phe
GXAHFRWDRFG

SEQ ID NO: 46 - protein, 13 aa, Artificial sequence
  Synthetic peptide
  MISC_FEATURE (2)..(2): D-Tyr
  MISC_FEATURE (4)..(4): Xaa = Nle
  MISC_FEATURE (5)..(5): D-Ala
  MISC_FEATURE (9)..(9): D-Trp
  MISC_FEATURE (11)..(11): D-Arg
GYVXAHFRWDRFG

SEQ ID NO: 47 - protein, 13 aa, Artificial sequence
  Synthetic peptide
  MISC_FEATURE (2)..(2): D-Tyr
  MISC_FEATURE (4)..(4): Xaa = Nle
  MISC_FEATURE (5)..(5): D-Ala
  MISC_FEATURE (9)..(9): D-Trp
GYVXAHFRWDRFG

SEQ ID NO: 48 - protein, 11 aa, Artificial sequence
  Synthetic peptide
  MISC_FEATURE (2)..(2): Xaa = Nle
  MISC_FEATURE (3)..(3): D-Ala
  MISC_FEATURE (7)..(7): D-Trp
  MISC_FEATURE (10)..(10): D-Phe
GXAHFRWDRFG

SEQ ID NO: 49 - protein, 12 aa, Artificial sequence
  Synthetic peptide
  MISC_FEATURE (3)..(3): Xaa = Nle
  MISC_FEATURE (8)..(8): D-Trp
YVXGHFRWDRFG

SEQ ID NO: 50 - protein, 12 aa, Artificial sequence
  Synthetic peptide
  MISC_FEATURE (3)..(3): Xaa = Nle
  MISC_FEATURE (6)..(6): D-Phe
  MISC_FEATURE (8)..(8): D-Trp
YVXGHFRWDRFG

SEQ ID NO: 51 - protein, 12 aa, Artificial sequence
  Synthetic peptide
  MISC_FEATURE (3)..(3): Xaa = Nle
  MISC_FEATURE (4)..(4): D-Ala
  MISC_FEATURE (6)..(6): D-Phe
  MISC_FEATURE (8)..(8): D-Trp
YVXAHFRWDRFG

SEQ ID NO: 52 - protein, 12 aa, Artificial sequence
  Synthetic peptide
  MISC_FEATURE (4)..(4): D-Ala
  MISC_FEATURE (6)..(6): D-Phe
  MISC_FEATURE (8)..(8): D-Trp
YVEAHFRWDRFG

SEQ ID NO: 53 - protein, 9 aa, Artificial sequence
  Synthetic peptide
  MISC_FEATURE (3)..(3): Xaa = Nle
  MISC_FEATURE (6)..(6): D-Phe
  MISC_FEATURE (8)..(8): D-Trp
YVXGHFRWD

SEQ ID NO: 54 - protein, 9 aa, Artificial sequence
  Synthetic peptide
  MISC_FEATURE (3)..(3): Xaa = Nle
  MISC_FEATURE (4)..(4): D-Ala
  MISC_FEATURE (6)..(6): D-Phe
  MISC_FEATURE (8)..(8): D-Trp
YVXAHFRWD

SEQ ID NO: 55 - protein, 7 aa, Artificial sequence
  Synthetic peptide
  MISC_FEATURE (6)..(6): D-Trp
SGHFRWD

SEQ ID NO: 56 - protein, 7 aa, Artificial sequence
  Synthetic peptide
  MISC_FEATURE (4)..(4): D-Phe
  MISC_FEATURE (6)..(6): D-Trp
SGHFRWD

SEQ ID NO: 57 - protein, 7 aa, Artificial sequence
  Synthetic peptide
  MISC_FEATURE (6)..(6): D-Trp
TGHFRWD

SEQ ID NO: 58 - protein, 7 aa, Artificial sequence
  Synthetic peptide
  MISC_FEATURE (4)..(4): D-Phe
  MISC_FEATURE (6)..(6): D-Trp
TGHFRWD

SEQ ID NO: 59 - protein, 12 aa, Artificial sequence
  Synthetic peptide
  MISC_FEATURE (3)..(3): Xaa = Nle
  MISC_FEATURE (4)..(4): D-Ala
  MISC_FEATURE (8)..(8): D-Trp
YVXAHFRWDRFG

SEQ ID NO: 60 - protein, 11 aa, Artificial sequence
  Synthetic peptide
  MISC_FEATURE (3)..(3): Xaa = Nle
  MISC_FEATURE (4)..(4): D-Ala
  MISC_FEATURE (8)..(8): D-Trp
YVXAHFRWDRF

SEQ ID NO: 61 - protein, 10 aa, Artificial sequence
  Synthetic peptide
  MISC_FEATURE (3)..(3): Xaa = Nle
  MISC_FEATURE (4)..(4): D-Ala
  MISC_FEATURE (8)..(8): D-Trp
YVXAHFRWDR

SEQ ID NO: 62 - protein, 7 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRWD

SEQ ID NO: 63 - protein, 12 aa, Artificial sequence
  Synthetic peptide
  MOD_RES (1)..(1): METHYLATION
  misc_feature (3)..(3): Xaa = Nle
  MISC_FEATURE (4)..(4): D-Ala
  MISC_FEATURE (8)..(8): D-Trp
YVXAHFRWDRFG

SEQ ID NO: 64 - protein, 12 aa, Artificial sequence
  Synthetic peptide
  MISC_FEATURE (3)..(3): Xaa = Nle
  MISC_FEATURE (4)..(4): D-Phe
  MISC_FEATURE (8)..(8): D-Trp
YVXFHFRWDRFG

SEQ ID NO: 65 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
  MISC_FEATURE (6)..(6): D-Arg
XAHFRWDRFG

SEQ ID NO: 66 - protein, 11 aa, Artificial sequence
  Synthetic peptide
  MISC_FEATURE (2)..(2): Xaa = Nle
  MISC_FEATURE (3)..(3): D-Ala
  MISC_FEATURE (7)..(7): D-Trp
VXAHFRWDRFG

SEQ ID NO: 67 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
  MISC_FEATURE (9)..(9): D-Phe
XAHFRWDRFK

SEQ ID NO: 68 - protein, 10 aa, Artificial sequence
  Synthetic peptide
  MISC_FEATURE (1)..(1): Xaa = Nle
  MISC_FEATURE (2)..(2): D-Val
  MISC_FEATURE (6)..(6): D-Trp
XVHFRWDRFG

SEQ ID NO: 69 - protein, 10 aa, Artificial sequence
  Synthetic peptide
  MISC_FEATURE (1)..(1): Xaa = Nle
  MISC_FEATURE (2)..(2): Xaa = D-Nle
  MISC_FEATURE (6)..(6): D-Trp
XXHFRWDRFG

SEQ ID NO: 70 - protein, 10 aa, Artificial sequence
  Synthetic peptide
  MISC_FEATURE (1)..(1): Xaa = Nle
  MISC_FEATURE (2)..(2): N-methyl-D-Ala
  MISC_FEATURE (6)..(6): D-Trp
XAHFRWDRFG

SEQ ID NO: 71 - protein, 10 aa, Artificial sequence
  Synthetic peptide
  MISC_FEATURE (1)..(1): Xaa = Nle
  MISC_FEATURE (2)..(2): D-Pro
  MISC_FEATURE (6)..(6): D-Trp
XPHFRWDRFG

SEQ ID NO: 72 - protein, 10 aa, Artificial sequence
  Synthetic peptide
  MISC_FEATURE (1)..(1): Xaa = Nle
  MISC_FEATURE (6)..(6): D-Trp
XGHFRWDRFG

SEQ ID NO: 73 - protein, 10 aa, Artificial sequence
  Synthetic peptide
  MISC_FEATURE (1)..(1): Xaa = Nle
  MISC_FEATURE (2)..(2): D-Ala
  MOD_RES (3)..(3): METHYLATION
  MISC_FEATURE (6)..(6): D-Trp
XAHFRWDRFG

SEQ ID NO: 74 - protein, 10 aa, Artificial sequence
  Synthetic peptide
  MISC_FEATURE (1)..(1): Xaa = Nle
  MISC_FEATURE (2)..(2): D-Ala
  MOD_RES (3)..(3): METHYLATION
  MISC_FEATURE (6)..(6): D-Trp
XAKFRWDRFG

SEQ ID NO: 75 - protein, 10 aa, Artificial sequence
  Synthetic peptide
  MISC_FEATURE (1)..(1): Xaa = Nle
  MISC_FEATURE (2)..(2): D-Ala
  MOD_RES (3)..(3): METHYLATION
  MISC_FEATURE (6)..(6): D-Trp
XAAFRWDRFG

SEQ ID NO: 76 - protein, 10 aa, Artificial sequence
  Synthetic peptide
  MISC_FEATURE (1)..(1): Xaa = Nle
  MISC_FEATURE (2)..(2): Xaa = D-alpha-Aminobutyric acid
  MISC_FEATURE (4)..(4): Phe(4-I)
  MISC_FEATURE (6)..(6): D-Trp
XXHFRWDRFG

SEQ ID NO: 77 - protein, 12 aa, Artificial sequence
  Synthetic peptide
  MISC_FEATURE (4)..(4): Xaa - D-alpha-Aminobutyric acid
  MISC_FEATURE (6)..(6): Phe(4-I)
  MISC_FEATURE (8)..(8): D-Trp
YVEXHFRWDRFG

SEQ ID NO: 78 - protein, 12 aa, Artificial sequence
  Synthetic peptide
  MISC_FEATURE (4)..(4): Xaa = 2-Aminoisobutyric acid
  MISC_FEATURE (6)..(6): Phe(4-I)
  MISC_FEATURE (8)..(8): D-Trp
YVEXHFRWDRFG

SEQ ID NO: 79 - protein, 10 aa, Artificial sequence
  Synthetic peptide
  MISC_FEATURE (1)..(1): Xaa = Nle
  MISC_FEATURE (2)..(2): D-Ala
  MISC_FEATURE (4)..(4): Phe(4-I)
  MISC_FEATURE (6)..(6): D-Trp
XARFRWDRFG

SEQ ID NO: 80 - protein, 10 aa, Artificial sequence
  Synthetic peptide
  MISC_FEATURE (1)..(1): Xaa = Nle
  MISC_FEATURE (2)..(2): D-Ala
  MISC_FEATURE (4)..(4): Phe(4-I)
  MISC_FEATURE (5)..(5): D-Arg
XAHFRWDRFG

SEQ ID NO: 81 - protein, 10 aa, Artificial sequence
  Synthetic peptide
  MISC_FEATURE (1)..(1): Xaa = Nle
  MISC_FEATURE (2)..(2): D-Ala
  MISC_FEATURE (4)..(4): Phe(4-I)
  MISC_FEATURE (6)..(6): Xaa = 3-(2-Naphthyl)-L-alanine
XAHFRXDRFG

SEQ ID NO: 82 - protein, 10 aa, Artificial sequence
  Synthetic peptide
  MISC_FEATURE (1)..(1): Xaa = Nle
  MISC_FEATURE (2)..(2): D-Ala
  MISC_FEATURE (4)..(4): Phe(4-I)
  MISC_FEATURE (6)..(6): D-Phe
XAHFRFDRFG

SEQ ID NO: 83 - protein, 10 aa, Artificial sequence
  Synthetic peptide
  MISC_FEATURE (1)..(1): Xaa = Nle
  MISC_FEATURE (2)..(2): D-Ala
  MISC_FEATURE (4)..(4): Phe(4-I)
  MISC_FEATURE (6)..(6): Xaa = 1,2,3,4-tetrahydroisoquinoline-3- carboxylic acid
XAHFRXDRFG

SEQ ID NO: 84 - protein, 10 aa, Artificial sequence
  Synthetic peptide
  MISC_FEATURE (1)..(1): Xaa = Nle
  MISC_FEATURE (2)..(2): D-Ala
  MISC_FEATURE (4)..(4): Phe(4-I)
  MISC_FEATURE (6)..(6): D-Phe
XAPFRFDRFG

SEQ ID NO: 85 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): alpha-Methyltryptophan
XAHFRWDRFG

SEQ ID NO: 86 - protein, 10 aa, Artificial sequence
  Synthetic peptide
  MISC_FEATURE (1)..(1): Xaa = Nle
  MISC_FEATURE (2)..(2): D-Ala
  MISC_FEATURE (4)..(4): Xaa = 3-(2-Naphthyl)-L-alanine
  MISC_FEATURE (6)..(6): D-Trp
XAHXRWDRFG

SEQ ID NO: 87 - protein, 10 aa, Artificial sequence
  Synthetic peptide
  MISC_FEATURE (1)..(1): Xaa = Nle
  MISC_FEATURE (2)..(2): D-Ala
  MISC_FEATURE (4)..(4): Phe(4-F)
  MISC_FEATURE (6)..(6): D-Trp
XAHFRWDRFG

SEQ ID NO: 88 - protein, 10 aa, Artificial sequence
  Synthetic peptide
  MISC_FEATURE (1)..(1): Xaa = Nle
  MISC_FEATURE (2)..(2): D-Ala
  MISC_FEATURE (4)..(4): Xaa = N-alpha-Fmoc-beta-(4-biphenyl)-L-alanine
  MISC_FEATURE (6)..(6): D-Trp
XAHXRWDRFG

SEQ ID NO: 89 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFKWDRFG

SEQ ID NO: 90 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRWARFG

SEQ ID NO: 91 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRWFRFG

SEQ ID NO: 92 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
  MISC_FEATURE (7)..(7): D-Phe
XAHFRWFRFG

SEQ ID NO: 93 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
  MISC_FEATURE (7)..(7): D-Phe
XAHFRWFRFG

SEQ ID NO: 94 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
  MISC_FEATURE (7)..(7): Xaa = Nle
XAHFRWXRFG

SEQ ID NO: 95 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRWKRFG

SEQ ID NO: 96 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRWDAFG

SEQ ID NO: 97 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRWDKFG

SEQ ID NO: 98 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRWDRPG

SEQ ID NO: 99 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRWDRAG

SEQ ID NO: 100 - protein, 10 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRWDRFV

SEQ ID NO: 101 - protein, 6 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRW

SEQ ID NO: 102 - protein, 7 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRWG

SEQ ID NO: 103 - protein, 7 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
XAHFRWF

SEQ ID NO: 104 - protein, 7 aa, Artificial sequence
  Synthetic peptide
  MISC_FEATURE (1)..(1): Xaa = Nle
  MISC_FEATURE (2)..(2): D-Ala
  MISC_FEATURE (6)..(6): D-Trp
  MISC_FEATURE (7)..(7): D-Phe
XAHFRWF

SEQ ID NO: 105 - protein, 7 aa, Artificial sequence
  Synthetic peptide
  MISC_FEATURE (1)..(1): Xaa = Nle
  MISC_FEATURE (2)..(2): D-Ala
  MISC_FEATURE (4)..(4): Phe(4-I)
  MISC_FEATURE (6)..(6): D-Trp
XAHFRWG

SEQ ID NO: 106 - protein, 7 aa, Artificial sequence
  Synthetic peptide
  MISC_FEATURE (1)..(1): Xaa = Nle
  MISC_FEATURE (2)..(2): Xaa = 2-Aminoisobutyric acid
  MISC_FEATURE (4)..(4): Phe(4-I)
  MISC_FEATURE (6)..(6): D-Trp
XXHFRWG

SEQ ID NO: 107 - protein, 11 aa, Artificial sequence
  Synthetic peptide
  MISC_FEATURE (7)..(7): D-Trp
VMGHFRWDRFG

SEQ ID NO: 108 - protein, 10 aa, Artificial sequence
  Synthetic peptide
  MISC_FEATURE (6)..(6): D-Trp
MGHFRWDRFG

SEQ ID NO: 109 - protein, 12 aa, Artificial sequence
  Synthetic peptide
  MISC_FEATURE (8)..(8): D-Trp
YVMGRFRWDRFG

SEQ ID NO: 110 - protein, 12 aa, Artificial sequence
  Synthetic peptide
  MISC_FEATURE (3)..(3): Xaa = Nle
  MISC_FEATURE (8)..(8): D-Trp
YVXGRFRWDRFG

(19) United States
(12) Patent Application Publication
(10) Pub. No.: US 2024/0287154 A1
(43) Pub. Date: Aug. 29, 2024

(54) MC3R AGONIST PEPTIDES

(71) Applicants: The Regents of the University of Michigan, Ann Arbor, MI (US); Courage Therapeutics, Inc., Newton, MA (US)

(72) Inventors: Savannah Y. WILLIAMS, Ann Arbor, MI (US); Luis E. GIMENEZ, Ann Arbor, MI (US); Tomi K. SAWYER, Newton, MA (US); Roger D. CONE, Ann Arbor, MI (US); Pil Heui LEE, Ann Arbor, MI (US); Patrick SWEENEY, Ann Arbor, MI (US)

(21) Appl. No.: 18/564,509

(22) PCT Filed: May 27, 2022
(86) PCT No.: PCT/US2022/031366

§ 371 Date: Nov. 27, 2023

Related U.S. Application Data
(60) Provisional application No. 63/193,950, filed on May 27, 2021.

(51) Int. Cl. C07K 14/68; A61K 38/00

(52) U.S. Cl. CPC C07K 14/68; A61K 38/00

(57) ABSTRACT

Provided herein are melanocortin 3 receptor (MC3R) agonist peptides and methods of use thereof for the treatment and/or prevention of eating disorders (e.g., anorexia nervosa, cachexia, etc.), metabolic disorders (e.g., sarcopenia), endocrine and growth disorders (e.g. delayed growth and/or delayed puberty), and/or emotional/mental disorders (e.g., depression, anxiety, OCD, PTSD, etc.). In particular, provided herein are MC3R agonist peptides that exhibit enhanced selectivity for MC3R over other melanocortin receptors (e.g., melanocortin 4 receptor (MC4R), melanocortin 1 receptor (MC1R), etc.) and/or are MC4R antagonists, and methods of use thereof. MC3R agonist peptides herein may exhibit enhanced in vitro potency, in vivo efficacy and pharmacokinetic properties compared to other know MC3R agonists.

### CROSS-REFERENCE TO RELATED APPLICATIONS

This application claims the benefit of U.S. Provisional Patent Application No. 63/193,950, filed on May 27, 2021, which is incorporated by reference herein.

### SEQUENCE LISTING

The text of the computer readable sequence listing filed herewith, titled “39119-601_SEQUENCE_LISTING_ST25”, created May 27, 2022, having a file size of 46,834 bytes, is hereby incorporated by reference in its entirety

### FIELD

Provided herein are melanocortin 3 receptor (MC3R) agonist peptides and methods of use thereof for the treatment and/or prevention of eating disorders (e.g., anorexia nervosa, cachexia, etc.), metabolic disorders (e.g., sarcopenia), endocrine and growth disorders (e.g. delayed growth and/or delayed puberty), and/or emotional/mental disorders (e.g., depression, anxiety, OCD, PTSD, etc.). In particular, provided herein are MC3R agonist peptides that exhibit enhanced selectivity for MC3R over other melanocortin receptors (e.g., melanocortin 4 receptor (MC4R), melanocortin 1 receptor (MC1R), etc.) and/or are MC4R antagonists, and methods of use thereof. MC3R agonist peptides herein may exhibit enhanced in vitro potency, in vivo efficacy and pharmacokinetic properties compared to other know MC3R agonists.

### BACKGROUND

Disorders of negative energy balance, such as anorexia nervosa and disease cachexia, are characterized by decreased food intake, dangerously low BMI, and an increased risk of anxiety and depression. Despite the severe consequences of these disorders, few pharmacological strategies exist to stimulate feeding and reduce anxiety in these at-risk patient populations. A large body of research has established the critical role of hypothalamic AgRP neural circuits in stimulating feeding, and reducing other competing motivational states including anxiety, fear, and alarm, and intense effort has focused on identifying pharmacological targets that suppress these circuits as potential therapeutics for obesity. However, the utility of pharmacological stimulation of these pathways in conditions of negative energy balance, such as anorexia nervosa or disease cachexia, has been much less studied.

### SUMMARY

Provided herein are melanocortin 3 receptor (MC3R) agonist peptides and methods of use thereof for the treatment and/or prevention of eating disorders (e.g., anorexia nervosa, cachexia, etc.), metabolic disorders (e.g., sarcopenia), endocrine and growth disorders (e.g. delayed growth and/or delayed puberty), and/or emotional/mental disorders (e.g., depression, anxiety, OCD, PTSD, etc.). In particular, provided herein are MC3R agonist peptides that exhibit enhanced selectivity for MC3R over other melanocortin receptors (e.g., melanocortin 4 receptor (MC4R), melanocortin 1 receptor (MC1R), etc.) and/or are MC4R antagonists, and methods of use thereof. MC3R agonist peptides herein may exhibit enhanced in vitro potency, in vivo efficacy and pharmacokinetic properties compared to other know MC3R agonists. In some embodiments, an MC3R peptide herein exhibits 70% or greater in vivo efficacy (e.g., >70%, >75%, >80%, >85%, >90%, >95%, etc.). In some embodiments, an MC3R peptide herein exhibits 10-fold or greater selectivity for MC3R over MC4R (e.g., 10-fold, 15-fold, 20-fold, 25-fold, 30-fold, 40-fold, 50-fold, 60-fold, 70-fold, 80-fold, 90-fold, 100-fold, or more or ranges therebetween). In some embodiments, an MC3R peptide herein exhibits 10-fold or greater selectivity for MC3R over MC1R (e.g., 10-fold, 15-fold, 20-fold, 25-fold, 30-fold, 40-fold, 50-fold, 60-fold, 70-fold, 80-fold, 90-fold, 100-fold, or more or ranges therebetween). In some embodiments, an MC3R peptide herein exhibits 10-fold or greater selectivity for MC3R over MC2R (e.g., 10-fold, 15-fold, 20-fold, 25-fold, 30-fold, 40-fold, 50-fold, 60-fold, 70-fold, 80-fold, 90-fold, 100-fold, or more or ranges therebetween). In some embodiments, an MC3R peptide herein exhibits 10-fold or greater selectivity for MC3R over MC5R (e.g., 10-fold, 15-fold, 20-fold, 25-fold, 30-fold, 40-fold, 50-fold, 60-fold, 70-fold, 80-fold, 90-fold, 100-fold, or more or ranges therebetween).

In some embodiments, provided herein are methods of treating an eating disorder comprising administering a melanocortin 3 receptor (MC3R) agonist to a subject suffering from the eating disorder. In some embodiments, the eating disorder is characterized by under eating. In some embodiments, the eating disorder is characterized by one or more emotional/mental symptoms. In some embodiments, the eating disorder is characterized by anxiety and/or depression. In some embodiments, the eating disorder is anorexia nervosa. In some embodiments, the eating disorder is Avoidant/Restrictive Food Intake Disorder (ARFID). In some embodiments, the eating disorder is cachexia. In some embodiments, the eating disorder is stress-induced anorexia. In some embodiments, the MC3R agonist is selective for MC3R over melanocortin 4 receptor (MC4R). In some embodiments, the MC3R agonist is a peptide. In some embodiments, the administration is repeated on a recurring basis for a period of at least 1 week (e.g., 1 week, 2 weeks, 1 month, 2 months, 4 months, 6 months, 9 months, 1 year, 2 years, 3, years, 4 years, or more). In some embodiments, the administration is repeated on a daily basis. In some embodiments, the administration is repeated on a twice-daily basis. In some embodiments, the administration is repeated on alternate days. In some embodiments, the administration is repeated on a weekly basis. In some embodiments, the administration is repeated on a recurring basis for a period of at least 1 month (e.g., 1 month, 2 months, 4 months, 6 months, 9 months, 1 year, 2 years, 3, years, 4 years, or more). In some embodiments, the administration is repeated on a recurring basis for a period of at least 1 year. In some embodiments, the MC3R agonist is co-administered with nutritional therapy, psychotherapy, nasogastric feeding, antidepressant agents, and/or antipsychotic agents.

In some embodiments, provided herein are methods of treating a metabolic disorder (e.g., sarcopenia) comprising administering a melanocortin 3 receptor (MC3R) agonist to a subject suffering from the metabolic disorder.

In some embodiments, provided herein are methods of treating or preventing delayed growth and/or delayed puberty comprising administering a melanocortin 3 receptor (MC3R) agonist to a subject suffering or at risk of delayed growth and/or delayed puberty.

In some embodiments, provided herein are methods of treating an emotional/mental disorder comprising administering a melanocortin 3 receptor (MC3R) agonist to a subject suffering from the emotional/mental disorder. In some embodiments, the emotional/mental disorder is characterized by anxiety and/or depression. In some embodiments, the MC3R agonist is peptide that is selective for MC3R over melanocortin 4 receptor (MC4R). In some embodiments, the administration is repeated on a recurring basis for a period of at least 1 week (e.g., 1 week, 2 weeks, 1 month, 2 months, 4 months, 6 months, 9 months, 1 year, 2 years, 3, years, 4 years, or more). In some embodiments, the administration is repeated on a daily basis. In some embodiments, the administration is repeated on a twice-daily basis. In some embodiments, the administration is repeated on a weekly basis. In some embodiments, the administration is repeated on a recurring basis for a period of at least 1 month (e.g., 1 month, 2 months, 4 months, 6 months, 9 months, 1 year, 2 years, 3, years, 4 years, or more). In some embodiments, the administration is repeated on a recurring basis for a period of at least 1 year. In some embodiments, the MC3R agonist is co-administered with psychotherapy (e.g., cognitive behavioral therapy, family therapy, etc.), antianxiety agents, mood stabilizers, stimulants, antidepressant agents, and/or antipsychotic agents.

In some embodiments, provided herein are compositions (e.g., pharmaceutical compositions) comprising a peptide having 4 or fewer (e.g., 4, 3, 2, 1, or 0) substitutions relative to the sequence:

(SEQ ID NO: 1)
X-AA0-AA1-AA2-AA3-AA4-AA5-AA6-AA7-AA8-AA9-AA10-
AA11-AA12-Y;

wherein X is a N-terminal cap moiety linked to the most N-terminal amino acid of the peptide and is acetyl, C18 diacid-Glu-PEG-Gly, Aryl(4-I)-PEG-Gly, or absent;
wherein AA0 is absent or Gly;
wherein AA1 is Tyr, D-Tyr, NMe-Tyr, or absent;
wherein AA2 is Val, Gly, Ala, Aib, or absent;
wherein AA3 is Met, Nle, Glu, Ser, Asp, homoGlu, or Thr;
wherein AA4 is Gly, D-Ala, D-Val, D-Nle, NMe-D-Ala, D-Pro, Ala, Aib, D-Abu, D-Phe, Glu, or absent;
wherein AA5 is His, Pro, Gln, Cit, NMe-His, NMe-Arg, NMe-Lys, NMe-Ala;
wherein AA6 is Phe, D-Phe, Nal(2′), Trp, D-Trp, Tic, Hph, Bip, D-Bip, aMe-Phe, D-Phe(4-NH—Ac), Phe(4-F), Phe(4tBu), Phe(4-Br), Phe(4-I), Phe(4-Cl), D-Phe(4-Br), D-Phe(4-I), NMe-Phe, or D-Phe(4-Cl);
wherein AA7 is Arg, D-Arg, Lys, Orn, Cit, or Nle;
wherein AA8 is aMe-D-Trp, D-Trp, Trp, D-Nal(2′), D-Phe, D-Tic, Tic, D-Phe, or D-Ala;
wherein AA9 is Asp, Ala, Phe, D-Phe, Nle, Lys, Gly, Orn, or absent;
wherein AA10 is Arg, D-Arg, Lys, Ala, or absent;
wherein AA11 is Phe, D-Phe, Pro, Gly, Ala, or absent;
wherein AA12 is Gly, Lys, Val, or absent;
wherein Y is a C-terminal cap linked to the most C-terminal amino acid of the peptide and is NH₂ or absent;
wherein AA2 is present if AA1 is present;
wherein AA2 and AA1 are present in AA0 is present;
wherein AA10 and AA11 are present if AA12 is present;
wherein AA10 is present if AA11 is present; and
wherein the peptide does not consist of Tyr-Val-Met-Gly-His-Phe-Arg-D-Trp-Asp-Arg-Phe-Gly (SEQ ID NO: 2).

In some embodiments, a peptide comprises AA5-AA8 of His-Phe (or a Phe variant amino acid)-Arg-D-Trp.

In some embodiments, provided herein are compositions (e.g., pharmaceutical compositions) comprising a peptide with 70% or greater (e.g., >70%, >75%, >80%, >85%, >90%, >95%, 100%) sequence similarity to the sequence:

(SEQ ID NO: 1)
X-AA0-AA1-AA2-AA3-AA4-AA5-AA6-AA7-AA8-AA9-AA10-
AA11-AA12-Y;

wherein X is a N-terminal cap moiety linked to the most N-terminal amino acid of the peptide and is acetyl, C18 diacid-Glu-PEG-Gly, Aryl(4-I)-PEG-Gly, or absent;
wherein AA0 is absent or Gly;
wherein AA1 is Tyr, D-Tyr, NMe-Tyr, or absent;
wherein AA2 is Val, Gly, Ala, Aib, or absent;
wherein AA3 is Met, Nle, Glu, Ser, Asp, homoGlu, or Thr;
wherein AA4 is Gly, D-Ala, D-Val, D-Nle, NMe-D-Ala, D-Pro, Ala, Aib, D-Abu, D-Phe, Glu, or absent;
wherein AA5 is His, Pro, Gln, Cit, NMe-His, NMe-Arg, NMe-Lys, NMe-Ala;
wherein AA6 is Phe, D-Phe, Nal(2′), Trp, D-Trp, Tic, Hph, Bip, D-Bip, aMe-Phe, D-Phe(4-NH—Ac), Phe(4-F), Phe(4tBu), Phe(4-Br), Phe(4-I), Phe(4-Cl), D-Phe(4-Br), D-Phe(4-I), NMe-Phe, or D-Phe(4-Cl);
wherein AA7 is Arg, D-Arg, Lys, Orn, Cit, or Nle;
wherein AA8 is aMe-D-Trp, D-Trp, Trp, D-Nal(2′), D-Phe, D-Tic, Tic, D-Phe, or D-Ala;
wherein AA9 is Asp, Ala, Phe, D-Phe, Nle, Lys, Gly, Orn, or absent;
wherein AA10 is Arg, D-Arg, Lys, Ala, or absent;
wherein AA11 is Phe, D-Phe, Pro, Gly, Ala, or absent;
wherein AA12 is Gly, Lys, Val, or absent;
wherein Y is a C-terminal cap linked to the most C-terminal amino acid of the peptide and is NH₂ or absent;
wherein AA2 is present if AA1 is present;
wherein AA2 and AA1 are present in AA0 is present;
wherein AA10 and AA11 are present if AA12 is present;
wherein AA10 is present if AA11 is present; and
wherein the peptide does not consist of Tyr-Val-Met-Gly-His-Phe-Arg-D-Trp-Asp-Arg-Phe-Gly (SEQ ID NO: 2).

In some embodiments, provided herein are compositions (e.g., pharmaceutical compositions) comprising a peptide of the sequence:

(SEQ ID NO: 1)
X-AA0-AA1-AA2-AA3-AA4-AA5-AA6-AA7-AA8-AA9-AA10-
AA11-AA12-Y;

wherein X is a N-terminal cap moiety linked to the most N-terminal amino acid of the peptide and is acetyl, C18 diacid-Glu-PEG-Gly, Aryl(4-I)-PEG-Gly, or absent;
wherein AA0 is absent or Gly;
wherein AA1 is Tyr, D-Tyr, NMe-Tyr, or absent;
wherein AA2 is Val, Gly, Ala, Aib, or absent;
wherein AA3 is Met, Nle, Glu, Ser, Asp, homoGlu, or Thr;
wherein AA4 is Gly, D-Ala, D-Val, D-Nle, NMe-D-Ala, D-Pro, Ala, Aib, D-Abu, D-Phe, Glu, or absent;
wherein AA5 is His, Pro, Gln, Cit, NMe-His, NMe-Arg, NMe-Lys, NMe-Ala;
wherein AA6 is Phe, D-Phe, Nal(2), Trp, D-Trp, Tic, Hph, Bip, D-Bip, aMe-Phe, D-Phe(4-NH—Ac), Phe(4-F), Phe(4tBu), Phe(4-Br), Phe(4-I), Phe(4-Cl), D-Phe(4-Br), D-Phe(4-I), NMe-Phe, or D-Phe(4-Cl);
wherein AA7 is Arg, D-Arg, Lys, Orn, Cit, or Nle;
wherein AA8 is aMe-D-Trp, D-Trp, Trp, D-Nal(2′), D-Phe, D-Tic, Tic, D-Phe, or D-Ala;
wherein AA9 is Asp, Ala, Phe, D-Phe, Nle, Lys, Gly, Orn, or absent;
wherein AA10 is Arg, D-Arg, Lys, Ala, or absent;
wherein AA11 is Phe, D-Phe, Pro, Gly, Ala, or absent;
wherein AA12 is Gly, Lys, Val, or absent;
wherein Y is a C-terminal cap linked to the most C-terminal amino acid of the peptide and is NH₂ or absent;
wherein AA2 is present if AA1 is present;
wherein AA2 and AA1 are present in AA0 is present;
wherein AA10 and AA11 are present if AA12 is present;
wherein AA10 is present if AA11 is present; and
wherein the peptide does not consist of Tyr-Val-Met-Gly-His-Phe-Arg-D-Trp-Asp-Arg-Phe-Gly (SEQ ID NO: 2).

In some embodiments, provided herein are compositions comprising a peptide having 1-4 substituted and/or terminally-deleted amino acids relative to the amino acid sequence Tyr-Val-Met-Gly-His-Phe-Arg-D-Trp-Asp-Arg-Phe-Gly (SEQ ID NO: 2). In some embodiments, provided herein are compositions comprising a peptide having 1-3 substitutions relative to the amino acid sequence Tyr-Val-Met-Gly-His-Phe-Arg-D-Trp-Asp (SEQ ID NO: 3). In some embodiments, provided herein are compositions comprising a peptide having 1-3 substitutions relative to the amino acid sequence Gly-His-Phe-Arg-D-Trp-Asp-Arg-Phe-Gly (SEQ ID NO: 4). In some embodiments, provided herein are compositions comprising a peptide having 1 or 2 or fewer substitutions relative to the amino acid sequence Gly-His-Phe-Arg-D-Trp-Asp (SEQ ID NO: 5). In some embodiments, provided herein are compositions comprising a peptide having 100% sequence similarity to one of SEQ ID NOS: 2-110. In some embodiments, provided herein are compositions comprising a peptide selected from one or SEQ ID NOS: 2-110. In some embodiments, the peptide is selected from one or SEQ ID NOS: 6-110.

In some embodiments, a peptide here comprises one or more non-proteinogenic amino acids or amino acid analogs. In some embodiments, a peptide herein is linear.

In some embodiments, provided herein are compositions (e.g., pharmaceutical compositions) comprising a peptide that is selective for binding melanocortin 3 receptor (MC3R) over melanocortin 4 receptor (MC4R). In some embodiments, the peptide is a melanocortin 3 receptor (MC3R) agonist.

In some embodiments, provided herein are methods of treating an eating disorder comprising administering a composition (e.g., pharmaceutical compositions) comprising a peptide herein to a subject suffering from the eating disorder. In some embodiments, the eating disorder is characterized by under eating. In some embodiments, the eating disorder is characterized by one or more emotional/mental symptoms. In some embodiments, the eating disorder is characterized by anxiety and/or depression. In some embodiments, the eating disorder is anorexia nervosa. In some embodiments, the composition is co-administered with nutritional therapy, psychotherapy, nasogastric feeding, antidepressant agents, and/or antipsychotic agents.

In some embodiments, provided herein are methods of treating an eating disorder comprising administering a composition (e.g., pharmaceutical compositions) comprising a peptide herein to a subject suffering from the emotional/mental disorder. In some embodiments, the emotional/mental disorder is characterized by anxiety and/or depression. In some embodiments, the composition is co-administered with psychotherapy, antianxiety agents, mood stabilizers, stimulants, antidepressant agents, and/or antipsychotic agents.

In some embodiments, methods are provided in which administration of a composition (e.g., pharmaceutical compositions) comprising a peptide herein is repeated on a recurring basis for a period of at least 1 week. In some embodiments, the administration is repeated on a daily basis. In some embodiments, the administration is repeated on a recurring basis for a period of at least 1 month. In some embodiments, the administration is repeated on a recurring basis for a period of at least 1 year.

In some embodiments, provided herein is the use of a composition (e.g., pharmaceutical compositions) comprising a peptide herein in the treatment or prevention of an eating disorder and/or emotional/mental disorder. In some embodiments, provided herein is the use of a composition (e.g., pharmaceutical compositions) comprising a peptide herein as a medicament. In some embodiments, provided herein is the use of a composition (e.g., pharmaceutical compositions) comprising a peptide herein the manufacture of a medicament.

### BRIEF DESCRIPTION OF THE DRAWINGS

FIG. 1. Exemplary results of pharmacological assay for MC3R agonist activity and specificity.

FIG. 2. Plasma stability of 10 MC3R agonists and positive control.

FIG. 3. Plasma stability of 5 MC3R agonists and positive control.

### Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “an MC3R agonist” is a reference to one or more MC3R agonists and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “MC3R agonist” refers to an agent (e.g., peptide, etc.) that binds to MC3R and activates MC3R to produce its biological activity. In some embodiments, an MC3R agonist binds to MC3R in the same location as a natural MC3R ligand (e.g., melanocyte-stimulating hormone and adrenocorticotropic hormone) and produce a functional response.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the term “anorexia nervosa” or synonymously “anorexia” a psychological condition characterized by a relentless desire to lose weight in the pursuit of thinness to the point of cachexia by voluntarily withholding foods and fluids, and, at times, by excessive exercising.

As used herein, the term “subject at risk for a disease,” for example, “a subject at risk for anorexia” or “a subject at risk for anxiety” refers to a subject with one or more risk factors for developing the disease (e.g., cancer). Depending upon the specific disease, risk factors may include, but are not limited to, gender, age, genetic predisposition, environmental exposures, infections, and previous incidents of diseases, lifestyle, etc.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., an MC3R agonist and one or more additional therapeutics) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

As used herein, the term “instructions for administering said compound to a subject,” and grammatical equivalents thereof, includes instructions for using the compositions contained in a kit for the treatment of conditions (e.g . . . providing dosing, route of administration, decision trees for treating physicians for correlating patient-specific characteristics with therapeutic courses of action).

The term “amino acid” refers to natural amino acids, unnatural amino acids, and amino acid analogs, all in their D and L stereoisomers, unless otherwise indicated, if their structures allow such stereoisomeric forms. Embodiments herein refer to various amino acid abbreviations (single-letter or three-letter abbreviations) that will be understood by those in the field. Any amino acid abbreviations not defined herein refer to their field-accepted meaning. For example, “NMe” preceding an amino acid name refers to an “N-methyl” group on the amino acid, “Nle” is “norleucine,” “Abu” is “a-Aminobutyric acid,” “Aib” is “2-Aminoisobutyric acid,” “Nal(2) is “3-(2-Naphthyl)-L-alanine,” “tic” is “1,2,3,4-tetrahydroisoquinoline-3-carboxylic acid,” “HpH” is “homophenylalanine,” “Bip” is “N-alpha-Fmoc-beta-(4-biphenyl)-L-alanine,” “D-Phe(4tBu)” is “D-4-tert-butyl-phenylalanine,” and the single-letter or three-letter abbreviations for the common proteinogenic amino acids are provided below.

The term “proteinogenic amino acids” refers to the 20 amino acids coded for in the human genetic code, and includes alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V). Selenocysteine and pyroolysine may also be considered proteinogenic amino acids

The term “non-proteinogenic amino acid” refers to an amino acid that is not naturally-encoded or found in the genetic code of any organism, and is not incorporated biosynthetically into proteins during translation. Non-proteinogenic amino acids may be “unnatural amino acids” (amino acids that do not occur in nature) or “naturally-occurring non-proteinogenic amino acids” (e.g., norvaline, ornithine, homocysteine, etc.). Examples of non-proteinogenic amino acids include, but are not limited to, azetidinecarboxylic acid, 2-aminoadipic acid, 3-aminoadipic acid, beta-alanine, naphthylalanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-alkylglycine including N-methylglycine, N-methylisoleucine, N-alkylpentylglycine including N-methylpentylglycine. N-methylvaline, naphthylalanine, norvaline, norleucine (“Norleu”), octylglycine, ornithine, pentylglycine, pipecolic acid, thioproline, homolysine, and homoarginine. Non-proteinogenic also include D-amino acid forms of any of the amino acids herein, as well as non-alpha amino acid forms of any of the amino acids herein (beta-amino acids, gamma-amino acids, delta-amino acids, etc.), all of which are in the scope herein and may be included in peptides herein.

The term “amino acid analog” refers to an amino acid (e.g., natural or unnatural, proteinogenic or non-proteinogenic) where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid: N-ethylglycine is an amino acid analog of glycine: or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers an oligomer to short polymer of amino acids linked together by peptide bonds. In contrast to other amino acid polymers (e.g., proteins, polypeptides, etc.), peptides are of about 30 amino acids or less in length. A peptide may comprise natural amino acids, non-natural amino acids, proteinogenic amino acids, non-proteinogenic amino acids, amino acid analogs, and/or modified amino acids. A peptide may be a subsequence of naturally occurring protein or a non-natural (artificial) sequence.

As used herein, the term “artificial” refers to compositions and systems that are designed or prepared synthetically, and are not naturally occurring. For example, an artificial peptide, peptoid, or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid I;
3) Asparagine (N) and Glutamine (Q);
4) Arginine I and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine I and Methionine (M).

Naturally occurring residues may be divided into classes based on common side chain properties, for example: polar positive (or basic) (histidine (H), lysine (K), and arginine I); polar negative (or acidic) (aspartic acid (D), glutamic acid I); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)): proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs. Non-conservative substitutions may involve the exchange of a member of one class for a member from another class.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any peptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 20 amino acids) may have up to X substitutions (e.g., 2) relative to SEQ ID NO:Z, and may therefore also be expressed as “having X (e.g., 2) or fewer substitutions relative to SEQ ID NO:Z.”

### DETAILED DESCRIPTION

Provided herein are melanocortin 3 receptor (MC3R) agonist peptides and methods of use thereof for the treatment and/or prevention of eating disorders (e.g., anorexia nervosa, cachexia, etc.), metabolic disorders (e.g., sarcopenia), endocrine and growth disorders (e.g. delayed growth and/or delayed puberty), and/or emotional/mental disorders (e.g., depression, anxiety, OCD, PTSD, etc.). In particular, provided herein are MC3R agonist peptides that exhibit enhanced selectivity for MC3R over other melanocortin receptors (e.g., melanocortin 4 receptor (MC4R), melanocortin 1 receptor (MC1R), etc.) and/or are MC4R antagonists, and methods of use thereof. MC3R agonist peptides herein may exhibit enhanced in vitro potency, in vivo efficacy and pharmacokinetic properties compared to other know MC3R agonists.

International Patent Application WO2020257662, which is incorporated by reference in its entirety, demonstrates and describes:that MC3R agonists stimulate feeding, increase body weight, and reduce anxiety in an AgRP neuron dependent manner; that MC3R is highly expressed in arcuate AgRP neurons, with significantly higher expression in these cells than anorexigenic POMC neurons: that MC3R agonist treatment phenocopies chemogenetic or optogenetic activation of ARC MC3R neurons, both stimulating feeding and body weight and reducing anxiety-related behavior: that chemogenetic inhibition of AgRP neuron reduces feeding and increases anxiety-related behavior: that test subjects lacking the MC3R display multiple behavioral phenotypes resembling anorexia nervosa, such as enhanced anxiety behavior and increased susceptibility to multiple forms of stress-induced anorexia; and that stimulation of the MC3R is a therapeutic approach for combating disorders at the intersection of energy metabolism and emotion, such as anorexia nervosa.

Central regulation of feeding and body weight is primarily controlled by neural circuits located in the hypothalamus and hindbrain (Refs. 1-3: herein incorporated by reference in their entireties). The central melanocortin system, composed of a set of two neuronal cell types located in the hypothalamic arcuate nucleus, the agouti related peptide neurons (AgRP neurons) and the pro-opiomelanocortin neurons (POMC neurons), engages this hypothalamic and hindbrain circuitry to potently regulate feeding and body weight (Refs. 4-6: herein incorporated by reference in their entireties). AgRP and POMC neurons project to largely overlapping brain regions to exert opposing effects on feeding and body weight. For example, AgRP neurons synthesize and release the melanocortin receptor antagonist/inverse agonist, agouti related peptide (AgRP), GABA, and neuropeptide Y to stimulate feeding and body weight (Ref. 7: herein incorporated by reference in its entirety). In contrast, POMC neurons synthesize and release the endogenous melanocortin receptor agonist, alpha melanocyte stimulating hormone (α-MSH), in addition to fast excitatory/inhibitory neurotransmitters to suppress feeding and reduce body weight (Refs. 4, 8: herein incorporated by reference in their entireties).

Hypothalamic AgRP neurons play a potent role in stimulating feeding (Refs. 6, 9, 10; herein incorporated by reference in their entireties). Ablation of AgRP neurons in adult mice leads to starvation and death, while stimulation rapidly and robustly stimulates food intake and body weight in sated animals (Refs. 11-13: herein incorporated by reference in their entireties). In addition to stimulating feeding, AgRP neuronal activation also suppresses competing need states, such as anxiety and fear, thereby promoting food seeking behavior in response to negative energy balance (Refs. 14-15: herein incorporated by reference in their entireties). Intense effort has focused on identifying pharmacological targets which suppress AgRP neural circuits as a potential therapeutic strategy for obesity.

MC3R is a G-protein coupled receptor primarily expressed within the brain, with particular dense expression observed in the hypothalamic arcuate nucleus (Refs. 16-17; herein incorporated by reference in their entireties). MC3R is expressed in AgRP neurons and recent studies suggest that MC3R has an important role in regulating the orexigenic activity of these cells (Ref. 16; herein incorporated by reference in its entirety). For example, MC3R knockout mice show multiple deficits in conditions that activate AgRP neurons, such as impaired feeding in response to a fast or caloric restriction (Refs. 18-20; herein incorporated by reference in their entireties). MC3R acts within presynaptic AgRP terminals in the paraventricular hypothalamus (PVN), promoting GABA release onto anorexigenic PVN melanocortin 4 receptor expressing neurons (Ref. 18: herein incorporated by reference in its entirety). Further, the MC3R plays a developmental role in growth and maturation to puberty (Ref. 62: incorporated by reference in its entirety).

Embodiments herein provide for the modulation (e.g., activation) of MC3R in order to achieve a desired impact on an eating disorder (e.g., anorexia, orthorexia, cachexia, etc.), eating habits (e.g., undereating, etc.), a metabolic abnormality (sarcopenia), a growth or endocrine disorder (delayed growth or delayed puberty), or a psychological condition (e.g., anxiety, depression, etc.), etc. In some embodiments, an MC3R agonist peptide for activating MC3R is administered to a subject and/or co-administered with one or more additional therapeutics/therapies.

In some embodiments, provided herein are methods of treating, preventing, and/or ameliorating the symptoms of eating disorders (e.g., anorexia nervosa, cachexia, etc.), metabolic disorders (e.g., sarcopenia), endocrine disorders (delayed growth and/or puberty), and/or emotional/mental disorders (e.g., depression, anxiety, etc.) by enhancing of the activity of MC3R in a subject via administration of a MC3R agonist peptide).

In some embodiments, the subject suffers from an eating disorder such as anorexia nervosa, bulimia nervosa, pica, rumination disorder, avoidant or restrictive food intake disorder, orthorexia nervosa, etc. In some embodiments, the subject suffers from anorexia. In some embodiments, the subject is at risk of developing an eating disorder (e.g., anorexia), having a recurrence of an eating disorder, relapsing into an eating disorder, or exhibiting physical symptoms of an eating disorder (e.g., low weight, restrictive eating, weight loss, etc.).

In some embodiments, provided herein are methods of treating a metabolic disorder (e.g., sarcopenia) comprising administering a melanocortin 3 receptor (MC3R) agonist to a subject suffering from the metabolic disorder.

In some embodiments, provided herein are methods of treating or preventing delayed growth and/or delayed puberty comprising administering a melanocortin 3 receptor (MC3R) agonist to a subject suffering or at risk of delayed growth and/or delayed puberty.

In some embodiments, the subject suffers from a psychological condition or mental illness such as anxiety depression, dipolar affective disorder, psychoses, obsessive compulsive disorder, post-traumatic stress disorder, etc. In some embodiments, the subject suffers from anxiety. In some embodiments, the subject is at risk of developing mental illness or exhibiting symptoms of a mental illness.

In some embodiments, provided herein are MC3R agonist peptides comprising sequence variants of [D-Trp8]-γ-MSH (SEQ ID NO: 2). In some embodiments, provided herein are peptides of the sequence X-AA0-AA1-AA2-AA3-AA4-AA5-AA6-AA7-AA8-AA9-AA10-AA11-AA12-Y (SEQ ID NO: 1): wherein X is a N-terminal cap moiety linked to the most N-terminal amino acid of the peptide and is acetyl, C18 diacid-Glu-PEG-Gly, Aryl(4-1)-PEG-Gly, or absent: wherein AA0 is absent or Gly: wherein AA1 is Tyr, D-Tyr, NMe-Tyr, or absent: wherein AA2 is Val, Gly, Ala, Aib, or absent: wherein AA3 is Met, Nle, Glu, Ser, Asp, homoGlu, or Thr: wherein AA4 is Gly, D-Ala, D-Val, D-Nle, NMe-D-Ala, D-Pro, Ala, Aib, D-Abu, D-Phe, Glu, or absent: wherein AA5 is His, Pro, Gln, Cit, NMe-His, NMe-Arg, NMe-Lys, NMe-Ala: wherein AA6 is Phe, D-Phe, Nal(2′), Trp, D-Trp, Tic, Hph, Bip, D-Bip, aMe-Phe, D-Phe(4-NH—Ac), Phe(4-F), Phe(4tBu), Phe(4-Br), Phe(4-I), Phe(4-Cl), D-Phe(4-Br), D-Phe(4-I), NMe-Phe, or D-Phe(4-Cl): wherein AA7 is Arg, D-Arg, Lys, Orn, Cit, or Nle: wherein AA8 is aMe-D-Trp, D-Trp, Trp, D-Nal(2′), D-Phe, D-Tic, Tic, D-Phe, or D-Ala: wherein AA9 is Asp, Ala, Phe, D-Phe, Nle, Lys, Gly, Orn, or absent: wherein AA10 is Arg, D-Arg, Lys, Ala, or absent: wherein AA11 is Phe, D-Phe, Pro, Gly, Ala, or absent: wherein AA12 is Gly, Lys, Val, or absent: wherein Y is a C-terminal cap linked to the most C-terminal amino acid of the peptide and is NH₂ or absent: wherein AA2 is present if AA1 is present: wherein AA2 and AA1 are present in AA0 is present: wherein AA10 and AA11 are present if AA12 is present: wherein AA10 is present if AA11 is present; and wherein the peptide does not consist of Tyr-Val-Met-Gly-His-Phe-Arg-D-Trp-Asp-Arg-Phe-Gly (SEQ ID NO: 2).

In some embodiments, provided herein are peptides having at least 70% (e.g., >70%, >75%, >80%, >85%, >90%, >95%, 100%) conservative sequence similarity with the sequence X-X-AA0-AA1-AA2-AA3-AA4-AA5-AA6-AA7-AA8-AA9-AA10-AA11-AA12-Y (SEQ ID NO: 1). In some embodiments, provided herein are peptides having at least 70% (e.g., >70%, >75%, >80%, >85%, >90%, >95%, 100%) semi-conservative sequence similarity with the sequence X-AA0-AA1-AA2-AA3-AA4-AA5-AA6-AA7-AA8-AA9-AA10-AA11-AA12-Y (SEQ ID NO: 1). In some embodiments, provided herein are peptides having at least 70% (e.g., >70%, >75%, >80%, >85%, >90%, >95%, 100%) sequence identity with the sequence X-AA0-AA1-AA2-AA3-AA4-AA5-AA6-AA7-AA8-AA9-AA10-AA11-AA12-Y (SEQ ID NO: 1).

In some embodiments, peptides herein have at least 1 (e.g., 1, 2, 3, 4, 5, or more) substitution or terminal deletion relative to SEQ ID NO: 2.

In some embodiments, provided herein are peptides having 4 or fewer (e.g., 4, 3, 2, 1) substitutions (e.g., conservative, semi-conservative, unconservative, etc.) relative to one or more of SEQ ID NOS: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, and 110.

In some embodiments, the N-terminal cap moiety linked to the most N-terminal amino acid of the peptide. In some embodiments, the N-terminal cap moiety and is an acetyl group. In some embodiments, the N-terminal cap moiety is a pharmacokinetic (PK) modifying group. Such groups are described in Refs. 59-61 (incorporated by reference in their entireties) and include groups comprising a PK-modifying moiety (e.g., 4-iodo phenyl, C18 diacid, etc.), an amino acid linker moiety (e.g., Gly, γGlu, etc.), and a PEG moiety (e.g., methoxy PEG (e.g., mPEG-2, mPEG-3, MPEG-4, mPEG-5, mPEG-6, or more)). In some embodiments, an N-terminal cap is of the general structure:

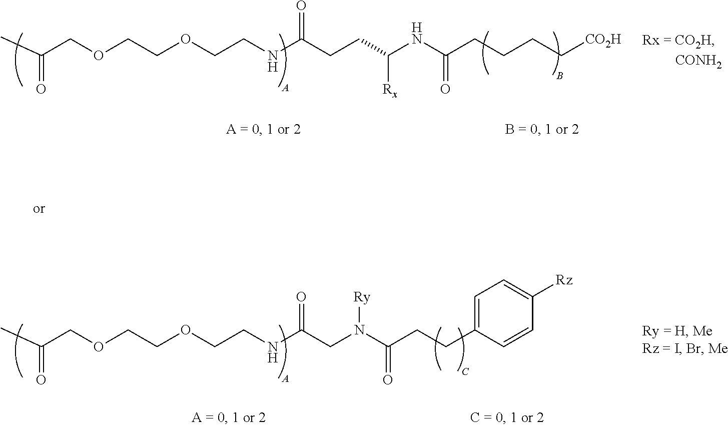

Examples of PK-modifying caps include C18 diacid-γGlu-mPEG2 (PKcap1) and Aryl(4-I)-Gly-mPEG2 (PKcap2):

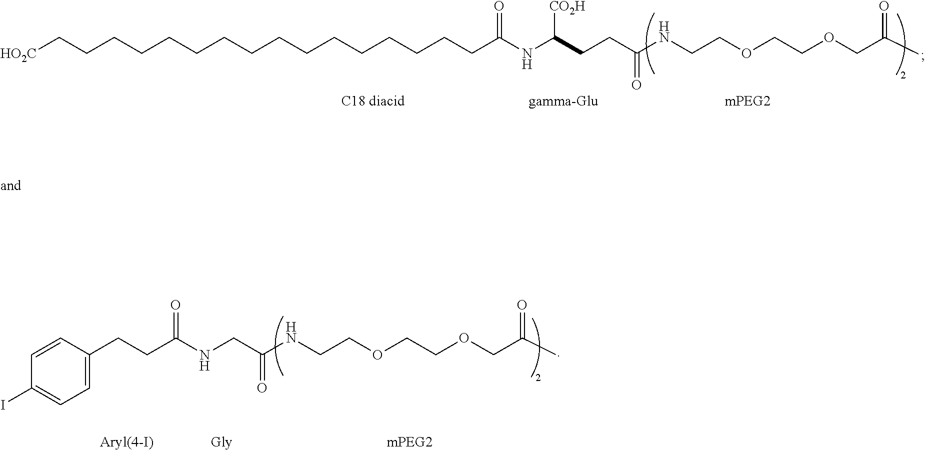

Other PK-modifying caps are within the scope herein.

In some embodiments, peptides herein comprise natural amino acids, unnatural amino acids, modified amino acids, non-proteinogenic amino acids, amino acid analogs, etc.

In some embodiments, a MC3R agonist peptide is administered to a subject (e.g., by any suitable route of administration and within any suitable pharmaceutical formulation). In some embodiments, the MC3R agonist peptide is selective for MC3R over MC4R. In some embodiments, the MC3R agonist peptide binds to MC3R in the subject. In some embodiments, the activity of MC3R is enhanced by the administration of the MC3R agonist peptide.

In some embodiments, methods herein comprise administering a MC3R agonist peptide to a subject at risk of and/or suffering from an eating disorder (e.g., anorexia) and/or a mental illness (e.g., anxiety). In some embodiments, administration of the MC3R agonist peptide results in increased eating, bodyweight, and/or reduced anxiety in the subject.

In some embodiments, the MC3R agonist peptide is administered locally. In some embodiments, the MC3R agonist peptide is administered systemically. In some embodiments, the MC3R agonist peptide is administered in a manner such that the MC3R agonist peptide reaches and/or localizes in the brain. In some embodiments, the MC3R agonist is administered in a manner such that the MC3R agonist peptide reaches and/or localizes in the hypothalamus. In some embodiments, the MC3R agonist peptide is administered in a manner such that the MC3R agonist reaches and/or localizes in AgRP neurons. In some embodiments, the MC3R agonist peptide is administered in a manner such that the [MC3R agonist peptide reaches and/or localizes in POMC neurons.

In some embodiments, a MC3R agonist peptide binds MC3R selectively over other melanocortin receptors (e.g., MC1R, MC2R, MC4R, MC5R). In some embodiments, a MC3R agonist peptide binds MC3R with an affinity that is at least 2-fold greater (e.g., 2×, 3×, 4×, 5×, 6×, 7×, 8×, 9×, 10×, 20×, 30×, 40×, 50×, 60×, 70×, 80×, 90×, 100×, 200×, 500×, 1000×, 2000×, 5000×, or more) than the binding affinity of the MC3R agonist peptide with one or more other melanocortin receptors (e.g., MC1R, MC2R, MC4R, MC5R). In some embodiments, a MC3R agonist peptide herein binds MC3R selectively over MC4R. In some embodiments, a MC3R agonist peptide binds MC3R with an affinity that is at least 2-fold greater (e.g., 2×, 3×, 4×, 5×, 6×, 7×, 8×, 9×, 10×, 20×, 30×, 40×, 50×, 60×, 70×, 80×, 90×, 100×, 200×, 500×, 1000×, 2000×, 5000×, or more) than the binding affinity of the MC3R agonist with MC4R.

In some embodiments, a MC3R agonist peptide enhances the activity of MC3R selectively over one or more other melanocortin receptors (e.g., MC1R, MC2R, MC4R, MC5R). In some embodiments, a MC3R agonist peptide enhances the activity of MC3R at least 2-fold greater (e.g., 2×, 3×, 4×, 5×, 6×, 7×, 8×, 9×, 10×, 20×, 30×, 40×, 50×, 60×, 70×, 80×, 90×, 100×, 200×, 500×, 1000×, 2000×, 5000×, or more) than one or more other melanocortin receptors (e.g., MC1R, MC2R, MC4R, MC5R). In some embodiments, a MC3R agonist peptide enhances the activity of MC3R selectively over MC4R. In some embodiments, a [MC3R agonist peptide enhances the activity of MC3R at least 2-fold greater (e.g., 2×, 3×, 4×, 5×, 6×, 7×, 8×, 9×, 10×, 20×, 30×, 40×, 50×, 60×, 70×, 80×, 90×, 100×, 200×, 500×, 1000×, 2000×, 5000×, or more) than MC4R.

In some embodiments, a MC3R agonist peptide is co-administered with an additional agent or therapy. In some embodiments, the co-administered agent is for the treatment or prevention of the same condition/disease/symptom as the MC3R agonist peptide (e.g., anorexia, anxiety, etc.). In some embodiments, the co-administered agent is for the treatment or prevention of a side-effect of the [D-Trp8]-y-MSH variant peptide. In some embodiments, the co-administered agent is for the treatment or prevention of a comorbidity not treated of prevented by the MC3R agonist peptide (e.g., bulimia, depression, obsessive-compulsive disorder, pain, etc.).

In some embodiments, the MC3R agonist peptide is co-administered with psychotherapy, nasogastric feeding, antidepressant agents, antianxiety agents, mood stabilizers, stimulants, and/or antipsychotic agents.

The term “psychotherapy” refers to use of non-pharmacological therapies a clinician or therapist uses any of a variety of techniques that involve verbal and other interactions with a patient to affect a positive therapeutic outcome. Such techniques include, but are not limited to, behavior therapy, cognitive therapy, psychodynamic therapy, psychoanalytic therapy, group therapy, family counseling, art therapy, music therapy, vocational therapy, humanistic therapy, existential therapy, transpersonal therapy, client-centered therapy (also called person-centered therapy), Gestalt therapy, biofeedback therapy, rational emotive behavioral therapy, reality therapy, response based therapy, Sandplay therapy, status dynamics therapy, hypnosis and validation therapy. Any suitable psychotherapy techniques, including those listed above, may be co-administered with a MC3R agonist peptide for the treatment/prevention of appropriate conditions/diseases (e.g., eating disorders (e.g., anorexia, cachexia, etc.), mental disease (e.g., anxiety, depression, etc.), etc.

Nasogastric (NG) feeding involves the use of a special tube (NG tube) that carries food to the stomach through the nose. In some embodiments, NG feeding is utilized in place of oral feeding and/or as a supplement to oral feeding, particularly in the earliest stage of treatment for an eating disorder. In some embodiments, NG feeding is co-administered with an MC3R agonist. In some embodiments, NG feeding is ceased once eating orally becomes sufficient to produce weight gain (e.g., as a result of the effect of the MC3R agonist).

In some embodiments, a MC3R agonist peptide is co-administered with an antidepressant agent. Suitable antidepressants for co-administration may include serotonin and noradrenaline reuptake inhibitors (e.g., duloxetine (Cymbalta), venlafaxine (Effexor), desvenlafaxine (Pristiq), etc.), selective serotonin reuptake inhibitors (e.g., italopram (Celexa), escitalopram (Lexapro), fluoxetine (Prozac, Sarafem), fluvoxamine (Luvox), paroxetine (Paxil), sertraline (Zoloft), etc.), tricyclic antidepressants (e.g., amitriptyline (Elavil), amoxapine-clomipramine (Anafranil), desipramine (Norpramin), doxepin (Sinequan), imipramine (Tofranil), nortriptyline (Pamelor), protriptyline (Vivactil), trimipramine (Surmontil), etc.), monoamine oxidase inhibitors (e.g., phenelzine (Nardil), tranylcypromine (Parnate), isocarboxazid (Marplan), selegiline (EMSAM, Eldepryl), etc.), noradrenaline and specific serotoninergic antidepressants (e.g., Mianserin (Tolvon), Mirtazapine (Remeron, Avanza, Zispin, etc.), etc.

In some embodiments, a MC3R agonist peptide is co-administered with an antianxiety agent. Suitable antianxiety medications for co-administration may include selective serotonin reuptake inhibitors, serotonin-norepinephrine reuptake inhibitors, tricyclics, benzodiazepines (e.g., alprazolam (Xanax), chlordiazepoxide (Librium), diazepam (Valium), lorazepam (Ativan) etc.), beta-blockers (e.g., atenolol (Tenormin), propranolol (Inderal), etc.), buspirone (BuSpar), monoamine oxidase inhibitors, etc.

In some embodiments, a MC3R agonist peptide is co-administered with a mood stabilizer. Suitable mood stabilizers for co-administration may include lithium, anticonvulsants (e.g., valproate, lamotrigine, carbamazepine, etc.), etc.

In some embodiments, a MC3R agonist peptide is co-administered with a stimulant. Suitable stimulants for co-administration may include amphetamine/dextroamphetamine (Adderall), dextroamphetamine (Dexedrine, ProCentra, Zenzedi), dexmethylphenidate (Focalin), methylphenidate (Ritalin), amphetamine sulfate (Evekeo), methylphenidate (Ritalin SR, Metadate ER, Methylin ER), amphetamine (Adzenys XR-ODT, Dyanavel XR), dexmethylphenidate (Focalin XR), dextroamphetamine (Adderall XR), lisdexamfetamine (Vyvanse), methylphenidate (Concerta, Daytrana, Jornay PM, Metadate CD, Quillivant XR, Quillichew ER, Ritalin LA), etc.

In some embodiments, a MC3R agonist peptide is co-administered with any agent or medication suitable for the treatment of the eating disorders and/or mental illnesses described herein.

In some embodiments, any suitable routes and/or modes of administering the agents herein (e.g., MC3R agonist peptide, co-administered agent, etc.) find use in embodiments herein. In some embodiments, the compositions and methods described herein act upon the central nervous system (CNS) and therefore routes and/or modes of administration that facilitate entry of the agents into the CNS are utilized. In some embodiments, the compositions and methods described herein act upon the brain of a subject and therefore routes and/or modes of administration that facilitate entry of the agents into the brain (e.g., allow agents to cross the blood-brain barrier) are utilized. In some embodiments, the compositions and methods described herein act upon the hypothalamus of a subject and therefore routes and/or modes of administration that facilitate delivery of the agents to the hypothalamus are utilized. In some embodiments, the compositions and methods described herein act upon the arcuate nucleus of the hypothalamus of a subject and therefore routes and/or modes of administration that facilitate delivery of the agents to the arcuate nucleus are utilized. In some embodiments, the compositions and methods described herein act upon the AgRP neurons of a subject and therefore routes and/or modes of administration that facilitate delivery of the agents to AgRP neurons are utilized. In some embodiments, the compositions and methods described herein act upon the POMC neurons of a subject and therefore routes and/or modes of administration that facilitate delivery of the agents to POMC neurons are utilized.

In some embodiments, routes of administration, formation of the desired agent, and the pharmaceutical composition are selected to provide efficient and effective delivery. In some embodiments, the therapeutic agents herein (e.g., MC3R agonist peptide, co-administered agent, etc.) are provided in pharmaceutical formulations for administration to a subject by a suitable route. The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. Moreover, the pharmaceutical compositions described herein (e.g., comprising a MC3R agonist peptide, a co-administered agent, etc.) are formulated into any suitable dosage form, including but not limited to, aqueous oral dispersions, liquids, gels, syrups, elixirs, slurries, suspensions, aerosols, fast melt formulations, effervescent formulations, lyophilized formulations, tablets, powders, pills, dragees, and capsules.

One may administer the compounds and/or compositions in a local rather than systemic manner, for example, via injection of the compound directly into an organ or tissue, often in a depot preparation or sustained release formulation. Such long acting formulations may be administered by implantation (for example subcutaneously or intramuscularly) or by intramuscular injection. Furthermore, one may administer the drug in a targeted drug delivery system, for example, in a liposome coated with organ-specific antibody. The liposomes will be targeted to and taken up selectively by the organ. In addition, the drug may be provided in the form of a rapid release formulation, in the form of an extended release formulation, or in the form of an intermediate release formulation.

Pharmaceutical preparations for oral use can be obtained by mixing one or more solid excipients with the therapeutic agent (e.g., MC3R agonist peptide, a co-administered agent, etc.) with any suitable substituents and functional groups disclosed herein, optionally grinding the resulting mixture, and processing the mixture of granules, after adding suitable auxiliaries, if desired, to obtain tablets, pills, or capsules. Suitable excipients include, for example, fillers such as sugars, including lactose, sucrose, mannitol, or sorbitol: cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose: or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

In some embodiments, agents are delivered by inhalation. For administration by inhalation, the agents described herein (e.g., MC3R agonist peptide, a co-administered agent, etc.) may be in a form as an aerosol, a mist or a powder. In some embodiments, pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Buccal formulations that include the agents described herein (e.g., MC3R agonist peptide, a co-administered agent, etc.) may be administered using a variety of formulations which include, but are not limited to, U.S. Pat. Nos. 4,229,447, 4,596,795, 4,755,386, and 5,739,136.

In some embodiments, the agents described herein (e.g., MC3R agonist peptide, a co-administered agent, etc.) are delivered transdermally. Transdermal formulations described herein may be administered using a variety of devices including but not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946, 144: incorporated by reference in their entireties.

In some embodiments, the agents described herein (e.g., MC3R agonist peptide, a co-administered agent, etc.) are delivered by parenteral administration (e.g., intramuscular, subcutaneous, intravenous, epidural, intracerebral, intracerebroventricular, etc.). Formulations suitable for parenteral administration may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Agents described herein (e.g., MC3R agonist peptide, a co-administered agent, etc.) may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally recognized in the field. For other parenteral injections, appropriate formulations may include aqueous or nonaqueous solutions, preferably with physiologically compatible buffers or excipients. Such excipients are generally recognized in the field.

In certain embodiments, delivery systems for pharmaceutical agents (e.g., MC3R agonist peptide, a co-administered agent, etc.) may be employed, such as, for example, liposomes and emulsions. In certain embodiments, compositions provided herein also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, an agent (e.g., MC3R agonist peptide, a co-administered agent, etc.) is administered in a therapeutically effective amount. Thus, a therapeutically effective amount is an amount that is capable of at least partially preventing or reversing a disease, disorder, or symptoms thereof. The dose required to obtain an effective amount may vary depending on the agent, formulation, disease or disorder, and individual to whom the agent is administered.

Determination of effective amounts may involve in vitro assays in which varying doses of agent are administered to cells in culture and the concentration of agent effective for ameliorating some or all symptoms is determined in order to calculate the concentration required in vivo. Effective amounts may also be based in in vivo animal studies.

Pharmaceutical compositions may be in unit dosage forms suitable for single administration of precise dosages. In unit dosage form, the formulation is divided into unit doses containing appropriate quantities of one or more agents (e.g., MC3R agonist peptide, a co-administered agent, etc.).

Dosing and administration regimes are tailored by the clinician, or others skilled in the pharmacological arts, based upon well-known pharmacological and therapeutic considerations including, but not limited to, the desired level of therapeutic effect, and the practical level of therapeutic effect obtainable.

In some embodiments, and upon the clinician's discretion, the administration of the compounds may be administered for an extended period of time, including throughout the duration of the patient's life in order to treat the disorder or ameliorate or otherwise control or limit the symptoms of the patient's disease.

In a case wherein the patient's status does improve, upon the clinician's discretion the administration of the agents (e.g., MC3R agonist peptide, a co-administered agent, etc.) may be given continuously: alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from about 10% to about 100%, including, by way of example only, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, once improvement of the patient's symptoms/disorder/condition has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

In some embodiments, the amount of a given agent that will correspond to such an amount will vary depending upon factors such as the particular compound, disease and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.02-about 5000 mg per day, in some embodiments, about 1-about 1500 mg per day. The desired dose may conveniently be presented in a single dose or as divided doses administered simultaneously (or over a short period of time) or at appropriate intervals, for example as two, three, four or more sub-doses per day.

As discussed above, provided in certain embodiments herein are combination therapies in which a MC3R agonist peptide is co-administered with an additional agent for the treatment of the disorder/condition, a side effect of the primary agent, or a comorbidity of the disorder/condition. Co-administered agents do not have to be administered in the same pharmaceutical composition, and may, because of different physical and chemical characteristics, have to be administered by different routes. Co-administered agents may be administered concurrently (in the same or separate formulations/compositions) or at separate times (separated by minutes, hours, days, etc.) The co-administered agents may be administered concurrently (e.g., simultaneously, essentially simultaneously or within the same treatment protocol) or sequentially, depending upon the nature of the disease, disorder, or condition, the condition of the patient, and the actual choice of agent used. The determination of the order of administration, and the number of repetitions of administration of each therapeutic agent during a treatment protocol, is well within the knowledge of the clinician after evaluation of the disease being treated and the condition of the patient.

Therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

For combination therapies described herein, dosages of the co-administered agents will of course vary depending on the type of co-drug employed, on the specific drug employed, on the disease being treated and so forth. In addition, when co-administered with one or more biologically active agents, the compound provided herein may be administered either simultaneously with the biologically active agent(s), or sequentially.

### EXPERIMENTAL

### Example 1

### Pharmacological In Vitro Assays

Determination of intracellular cAMP levels in live cells: A genetically-encoded CAMP split-luciferase reporter stably-expressing cell line (Promega, Madison, WI)(Binkowski et al., 2011, ACS chemical biology 6, 1193-1197.: incorporated by reference in its entirety) was used as the basis for the generation of stable clones expressing the human MC4R receptor (a gift from Promega), or the human MC3R (generated in-house by clonal selection). The stable cell lines were grown and maintained in selection media consisting of Dulbecco's modified Eagle media (DMEM) with 4.5 g/l D-glucose, and 4 mM L-glutamine (Thermo Fisher Scientific, Waltham, MA), supplemented with 10% fetal bovine serum, 100 units/ml penicillin, 100 μg/ml streptomycin, 2.5 μg/ml amphotericin B, 200 μg/ml hygromycin B (for positive selection of the GScAMP22f luciferase reporter), and Geneticin™ (G418) 700 μg/ml (for MC3R or MC4R selection). Actual serum concentration during the assay is estimated to be about 1%. Cell line identity is routinely verified by qPCR and MC3R- and MC4R-specific oligonucleotides.

The assay for the determination of the CAMP response in live cells was described previously. (Yu et al., 2020, Science 368, 428-433: incorporated by reference in its entirety). Ccells were seeded at a density of 20,000 cells per well using 384-well poly-D lysine-coated, clear bottom, and black-wall assay plates (Corning Inc. Corning, NJ). Cells were allowed to attach to the plates for 18 to 24 h after which growth media was removed and 20 ul of 4% D-luciferin (Promega) in CO2 independent, serum-free medium (Thermo Fisher Scientific) was added to each well. The luciferase substrate was allowed to permeate the cells for 120 min at 37° C. Intracellular cAMP levels were measured using an FDSS 7000EX Functional Drug Screening System (Hamamatsu Photonics, Hamamatsu, Japan) in the Center for Chemical Genomics at the Life Sciences Institute. This instrument allowed the in-line addition of test-peptides and receptor agonists while simultaneously acquiring the luminescence signal from live cells. Assay read steps were set as follows: baseline acquisition of 2 min, the addition of 10 μl of varying 3× concentrations of test-peptides or vehicle followed by 11 min measurement (measurement window 1), and 10 μl addition of 4× concentration of the endogenous melanocortin agonist α-MSH (Bachem, Bubendorf, Switzerland) followed by an additional 11 min response measurement (measurement window 2). The resulting final concentration of α-MSH was close to the respective receptor EC90 dose for each receptor. Intraplate concentration response curves for α-MSH and SHU-9119 (Phoenix Pharmaceuticals, Burlingame CA) were included as reference controls. A submaximal forskolin (20 μM) concentration was also included to serve as a normalization reference to account for cell number variations and differences in assay transducer efficiency between cell lines.

With this set up it was possible to evaluate the direct agonist effect of the test-peptides on the MC3R and MC4R cell lines during measurement window 1, while the antagonist profile in the presence of EC90 α-MSH was determined on measurement window 2. For data analysis baseline luminescence (i.e. the maximum luminescence signal from the initial 0 to 2 min window) was subtracted from the maximum luminescence obtained during measurement window 1 (2 to 13 min) and measurement window 2 (13 to 24 min) to yield the test-peptide elicited responses. EC50 or IC50 potency values were determined by non-linear regression by fitting the data to a sigmoid four-parameter variable slope model using the GraphPad Prism version 8.4 software package (San Diego CA).

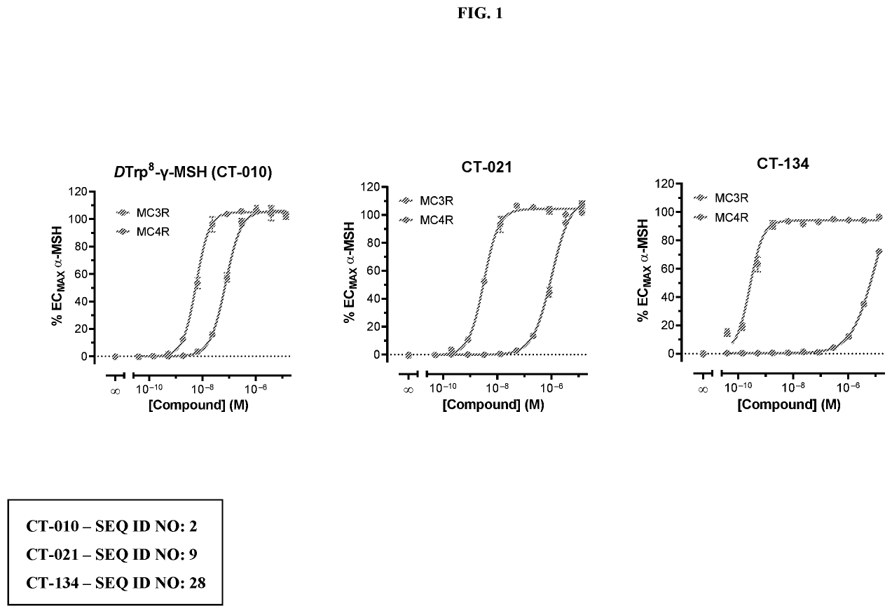

Exemplary results of pharmacological in vitro assays are provided in FIG. 1 and Table 1.

TABLE 1
MC3R cAMP (EC50) and MC3R selectivity relative to MC4R for exemplary
compounds

| | | | | | | MC3R |
|---|---|---|---|---|---|---|
| | | | | | MC3R | selectivity |
| Peptide | N-term. | | SEQ ID | C-term. | CAMP | (relative to |
| code | Mod. | Sequence | NO. | Mod. | (EC50) | MC4R) |
| CTX- | H | Tyr-Val-Met-Gly- | 2 | OH | 1.7 nM | 16.8 |
| 1100 | | His-Phe-Arg-D-Trp- | | | | |
| | | Asp-Arg-Phe-Gly | | | | |
| CTX- | H | Tyr-Val-Nle-D-Ala- | 3 | OH | 4.3 nM | 229 |
| 1103 | | His-Phe-Arg-D-Trp- | | | | |
| | | Asp-Arg-Phe-Gly | | | | |
| CTX- | H | Tyr-Val-Nle-D-Ala- | 4 | NH2 | 12 nM | 79 |
| 1108 | | His-Phe-Arg-D-Trp- | | | | |
| | | Asp | | | | |
| CTX- | Ac | Nle-D-Ala-His-Phe- | 5 | OH | 1.2 nM | 7,703 |
| 1122 | | Arg-D-Trp-Asp-Arg- | | | | |
| | | Phe-Gly | | | | |
| CTX- | Ac | Val-Nle-D-Ala-His- | 6 | OH | 3.1 nM | 2,123 |
| 1121 | | Phe-Arg-D-Trp-Asp- | | | | |
| | | Arg-Phe-Gly | | | | |
| CTX- | H | D-Tyr-Val-Nle-D- | 7 | OH | 1.7 nM | 747 |
| 1125 | | Ala-His-Phe-Arg-D- | | | | |
| | | Trp-Asp-Arg-Phe- | | | | |
| | | Gly | | | | |
| CTX- | H | Tyr-Val-Nle-Ala- | 8 | OH | 1.4 nM | 7.4 |
| 1133 | | His-Phe-Arg-D-Trp- | | | | |
| | | Asp-Arg-Phe-Gly | | | | |
| CTX- | H | Tyr-Val-Nle-Ala- | 9 | NH2 | 1.2 nM | 7.5 |
| 1129 | | His-Phe-Arg-D-Trp- | | | | |
| | | Asp | | | | |
| CTX- | H | Tyr-Val-Nle-D-Abu- | 10 | OH | 1 nM | 667 |
| 1135 | | His-Phe-Arg-D-Trp- | | | | |
| | | Asp-Arg-Phe-Gly | | | | |
| CTX- | H | Tyr-Val-Nle-D-Abu- | 11 | NH2 | 6.6 nM | 65 |
| 1131 | | His-Phe-Arg-D-Trp- | | | | |
| | | Asp | | | | |
| CTX- | H | Tyr-Val-Nle-D-Phe- | 12 | NH2 | 3.9 nM | 6.8 |
| 1132 | | His-Phe-Arg-D-Trp- | | | | |
| | | Asp | | | | |
| CTX- | H | Tyr-Val-Nle-Aib- | 13 | OH | 2.5 nM | 566 |
| 1134 | | His-Phe-Arg-D-Trp- | | | | |
| | | Asp-Arg-Phe-Gly | | | | |
| CTX- | H | Tyr-Val-Nle-Aib- | 14 | NH2 | 26.5 nM | 58 |
| 1130 | | His-Phe-Arg-D-Trp- | | | | |
| | | Asp | | | | |
| CTX- | H | D-Tyr-Val-Nle-D- | 15 | NH2 | 36 nM | 54 |
| 1142 | | Ala-His-Phe-Arg-D- | | | | |
| | | Trp-Asp | | | | |
| CTX- | H | NMe-Tyr-Val-Nle- | 16 | NH2 | 2.2 nM | 55 |
| 1141 | | D-Ala-His-Phe-Arg- | | | | |
| | | D-Trp-Asp | | | | |
| CTX- | H | Tyr-Val-Nle-D-Ala- | 17 | OH | 3 nM | 43 |
| 1126 | | His-Phe-Arg-D-Trp- | | | | |
| | | Asp-D-Arg-Phe-Gly | | | | |
| CTX- | H | Tyr-Val-Nle-D-Ala- | 18 | OH | 2.5 nM | 95 |
| 1127 | | His-Phe-Arg-D-Trp- | | | | |
| | | Asp-Arg-D-Phe-Gly | | | | |
| CTX- | H | Tyr-Val-Glu-Gly- | 19 | NH2 | 20 nM | 500 |
| 1105 | | His-Phe-Arg-D-Trp- | | | | |
| | | Asp-Arg-Phe-Gly | | | | |
| CTX- | H | Tyr-Val-Nle-D-Ala- | 20 | OH | 2.3 nM | 173 |
| 2120 | | His-Phe(4-Br)-Arg- | | | | |
| | | D-Trp-Asp-Arg-Phe- | | | | |
| | | Gly | | | | |
| CTX- | H | Tyr-Val-Nle-D-Ala- | 21 | OH | 2.5nM | 93 |
| 2121 | | His-Phe(4-Cl)-Arg- | | | | |
| | | D-Trp-Asp-Arg-Phe- | | | | |
| | | Gly | | | | |
| CTX- | H | Tyr-Val-Nle-D-Ala- | 22 | OH | 3.2nM | 324 |
| 2122 | | His-Phe(4-I)-Arg-D- | | | | |
| | | Trp-Asp-Arg-Phe- | | | | |
| | | Gly | | | | |
| CTX- | Ac | Nle-D-Ala-His-Phe- | 23 | OH | 52.9 nm | 535 |
| 1145 | | Arg-D-Trp-Asp-Arg- | | | | |
| | | D-Phe-Gly | | | | |
| CTX- | Ac | Nle-D-Ala-His-Phe- | 24 | OH | 170 nm | 1082 |
| 1146 | | Arg-D-Trp-Asp-D- | | | | |
| | | Arg-Phe-Gly | | | | |
| CTX- | Ac | Nle-D-Ala-His-Phe- | 25 | NH2 | 2.6 nm | 535 |
| 1158 | | Arg-D-Trp-Asp-Arg- | | | | |
| | | Phe | | | | |
| CTX- | Ac | Nle-D-Ala-His-Phe- | 26 | NH2 | 14.3 nm | 213 |
| 1159 | | Arg-D-Trp-Asp-Arg- | | | | |
| | | D-Phe | | | | |
| CTX- | Ac | Nle-D-Ala-His-Phe- | 27 | OH | 9.8 nm | 1,257 |
| 1160 | | Arg-D-Trp-Asp-Arg- | | | | |
| | | D-Phe-Lys | | | | |
| CTX- | Ac | Nle-D-Ala-His- | 28 | OH | 4.8 nm | 937 |
| 1167 | | Phe(4-I)-Arg-D-Trp- | | | | |
| | | Asp-Arg-Phe-Gly | | | | |
| CTX- | Ac | Nle-D-Ala-His- | 29 | NH2 | 4.8 nm | 200 |
| 1169 | | Phe(4-I)-Arg-D-Trp- | | | | |
| | | Asp-Arg-Phe | | | | |
| CTX- | Ac | Nle-D-Ala-His- | 30 | NH2 | 26.0 nm | 67 |
| 1170 | | Phe(4-I)-Arg-D-Trp- | | | | |
| | | Asp-Arg | | | | |
| CTX- | Ac | Nle-Aib-His-Phe- | 31 | OH | 13.4 nM | 3544 |
| 1143 | | Arg-D-Trp-Asp-Arg- | | | | |
| | | Phe-Gly | | | | |
| CTX- | Ac | Nle-D-Abu-His-Phe- | 32 | OH | 6.6 nm | 2,633 |
| 1144 | | Arg-D-Trp-Asp-Arg- | | | | |
| | | Phe-Gly | | | | |
| CTX- | Ac | Nle-D-Ala-His-Phe- | 33 | NH2 | 19.4 nm | 282 |
| 1163 | | Arg-D-Trp-Asp-Arg | | | | |
| CTX- | Ac | Nle-D-Ala-His-Phe- | 34 | NH2 | 1.5 nM | 83 |
| 1164 | | Arg-D-Trp-Gly-Lys- | | | | |
| | | Pro-Val | | | | |
| CTX- | Ac | Nle-D-Ala-His- | 35 | OH | 3.0 nM | 5598 |
| 1165 | | Phe(4-Br)-Arg-D- | | | | |
| | | Trp-Asp-Arg-Phe- | | | | |
| | | Gly | | | | |
| CTX- | Ac | Nle-D-Ala-His- | 36 | OH | 1.6 nM | 3099 |
| 1166 | | Phe(4-Cl)-Arg-D- | | | | |
| | | Trp-Asp-Arg-Phe- | | | | |
| | | Gly | | | | |
| CTX- | Ac | Nle-D-Ala-His-Bip- | 37 | NH2 | 28.6 nM | 485 |
| 1168 | | Arg-D-Trp-Asp-Arg- | | | | |
| | | Phe-Gly | | | | |
| CTX- | H | D-Tyr-Val-Nle-D- | 38 | NH2 | 54.0 nM | 234 |
| 1171 | | Ala-His-Phe(4-I)- | | | | |
| | | Arg-D-Trp-Asp | | | | |
| CTX- | PKcap1 | Gly-Nle-D-Ala-His- | 39 | OH | 556 nM | 335 |
| 1147 | | Phe-Arg-D-Trp-Asp- | | | | |
| | | Arg-Phe-Gly | | | | |
| CTX- | PKcap1 | Gly-Val Nle-D-Ala- | 40 | OH | 313 nM | 215 |
| 1149 | | His-Phe-Arg-D-Trp- | | | | |
| | | Asp-Arg-Phe-Gly | | | | |
| CTX- | PKcap1 | Gly-D-Tyr-Val Nle- | 41 | OH | 3133 nM | 415 |
| 1155 | | D-Ala-His-Phe-Arg- | | | | |
| | | D-Trp-Asp-D-Arg- | | | | |
| | | Phe-Gly | | | | |
| CTX- | PKcap2 | Gly-Nle-D-Ala-His- | 42 | OH | 1.5 nM | 1197 |
| 1148 | | Phe-Arg-D-Trp-Asp- | | | | |
| | | Arg-Phe-Gly | | | | |
| CTX- | PKcap2 | Gly-Val-Nle-D-Ala- | 43 | OH | 2.9 nM | 577 |
| 1150 | | His-Phe-Arg-D-Trp- | | | | |
| | | Asp-Arg-Phe-Gly | | | | |
| CTX- | PKcap2 | Gly-D-Tyr-Val-Nle- | 44 | OH | 2.1 nM | 419 |
| 1152 | | D-Ala-His-Phe-Arg- | | | | |
| | | D-Trp-Asp-Arg-Phe- | | | | |
| | | Gly | | | | |
| CTX- | PKcap2 | Gly-Nle-D-Ala-His- | 45 | OH | 5.8 nM | 1070 |
| 1154 | | Phe-Arg-D-Trp-Asp- | | | | |
| | | Arg-D-Phe-Gly | | | | |
| CTX- | PKcap2 | Gly-D-Tyr-Val Nle- | 46 | OH | 8.6 nM | 86 |
| 1156 | | D-Ala-His-Phe-Arg- | | | | |
| | | D-Trp-Asp-D-Arg- | | | | |
| | | Phe-Gly | | | | |
| CTX- | PKcap1 | Gly-D-Tyr-Val Nle- | 47 | OH | 299 nM | 126 |
| 1151 | | D-Ala-His-Phe-Arg- | | | | |
| | | D-Trp-Asp-Arg-Phe- | | | | |
| | | Gly | | | | |
| CTX- | PKcap1 | Gly-Nle-D-Ala-His- | 48 | OH | 5117 nM | 0.13 |
| 1153 | | Phe-Arg-D-Trp-Asp- | | | | |
| | | Arg-D-Phe-Gly | | | | |
| CTX- | H | Tyr-Val-Nle-Gly- | 49 | OH | 2.1 nM | 9 |
| 1101 | | His-Phe-Arg-D-Trp- | | | | |
| | | Asp-Arg-Phe-Gly | | | | |
| CTX- | H | Tyr-Val-Nle-Gly- | 50 | OH | 5 nM | 3 |
| 1102 | | His-D-Phe-Arg-D- | | | | |
| | | Trp-Asp-Arg-Phe- | | | | |
| | | Gly | | | | |
| CTX- | H | Tyr-Val-Nle-D-Ala- | 51 | OH | 3.5 nM | 27 |
| 1104 | | His-D-Phe-Arg-D- | | | | |
| | | Trp-Asp-Arg-Phe- | | | | |
| | | Gly | | | | |
| CTX- | H | Tyr-Val-Glu-D-Ala- | 52 | OH | 5.8 nM | 120 |
| 1106 | | His-D-Phe-Arg-D- | | | | |
| | | Trp-Asp-Arg-Phe- | | | | |
| | | Gly | | | | |
| CTX- | H | Tyr-Val-Nle-Gly- | 53 | NH2 | 4.4 nM | 2.4 |
| 1107 | | His-D-Phe-Arg-D- | | | | |
| | | Trp-Asp | | | | |
| CTX- | H | Tyr-Val-Nle-D-Ala- | 54 | NH2 | 13.1 nM | 10.6 |
| 1109 | | His-D-Phe-Arg-D- | | | | |
| | | Trp-Asp | | | | |
| CTX- | Ac | Ser-Gly-His-Phe- | 55 | NH2 | 7,396 nM | |
| 1110 | | Arg-D-Trp-Asp | | | | |
| CTX- | Ac | Ser-Gly-His-D-Phe- | 56 | NH2 | 17,100 nM | 0.1 |
| 1111 | | Arg-D-Trp-Asp | | | | |
| CTX- | Ac | Thr-Gly-His-Phe- | 57 | NH2 | 4,093 nM | 8.6 |
| 1112 | | Arg-D-Trp-Asp | | | | |
| CTX- | Ac | Thr-Gly-His-D-Phe- | 58 | NH2 | 4,426 nM | 0.4 |
| 1113 | | Arg-D-Trp-Asp | | | | |
| CTX- | H | Tyr-Val-Nle-D-Ala- | 59 | NH2 | 1.2 nM | 59 |
| 1118 | | His-Phe-Arg-D-Trp- | | | | |
| | | Asp-Arg-Phe-Gly | | | | |
| CTX- | H | Tyr-Val-Nle-D-Ala- | 60 | NH2 | 1.9 nM | 24 |
| 1119 | | His-Phe-Arg-D-Trp- | | | | |
| | | Asp-Arg-Phe | | | | |
| CTX- | H | Tyr-Val-Nle-D-Ala- | 61 | NH2 | 2.1 nM | 38 |
| 1120 | | His-Phe-Arg-D-Trp- | | | | |
| | | Asp-Arg | | | | |
| CTX- | Ac | Nle-D-Ala-His-Phe- | 62 | NH2 | 213 nM | 28 |
| 1123 | | Arg-D-Trp-Asp | | | | |
| CTX- | H | NMe-Tyr-Val-Nle- | 63 | OH | 1.7 nM | 69 |
| 1124 | | D-Ala-His-Phe-Arg- | | | | |
| | | D-Trp-Asp-Arg-Phe- | | | | |
| | | Gly | | | | |
| CTX- | H | Tyr-Val-Nle-D-Phe- | 64 | OH | 1.9 nM | 37 |
| 1136 | | His-Phe-Arg-D-Trp- | | | | |
| | | Asp-Arg-Phe-Gly | | | | |
| CTX- | Ac | Nle-D-Ala-His-Phe- | 65 | OH | 170 nM | 108 |
| 1146 | | Arg-D-Trp-Asp-D- | | | | |
| | | Arg-Phe-Gly | | | | |
| CTX- | R1 | Val-Nle-D-Ala-His- | 66 | OH | 710 nM | 6,796 |
| 1149 | | Phe-Arg-D-Trp-Asp- | | | | |
| | | Arg-Phe-Gly | | | | |
| CTX- | Ac | Nle-D-Ala-His-Phe- | 67 | OH | 952 nM | 22 |
| 1161 | | Arg-D-Trp-Asp-Arg- | | | | |
| | | D-Phe-Lys(R1) | | | | |
| CTX- | Ac | Nle-D-Val-His-Phe- | 68 | OH | 102 nM | 718 |
| 1181 | | Arg-D-Trp-Asp-Arg- | | | | |
| | | Phe-Gly | | | | |
| CTX- | Ac | Nle-D-Nle-His-Phe- | 69 | OH | 14.6 nM | 1406 |
| 1182 | | Arg-D-Trp-Asp-Arg- | | | | |
| | | Phe-Gly | | | | |
| CTX- | Ac | Nle-NMe-D-Ala- | 70 | OH | 71.6 nM | 515 |
| 1183 | | His-Phe-Arg-D-Trp- | | | | |
| | | Asp-Arg-Phe-Gly | | | | |
| CTX- | Ac | Nle-D-Pro-His-Phe- | 71 | OH | 607 nM | 28 |
| 1184 | | Arg-D-Trp-Asp-Arg- | | | | |
| | | Phe-Gly | | | | |
| CTX- | Ac | Nle-Gly-His-Phe- | 72 | OH | 2.7 nM | 2,312 |
| 1185 | | Arg-D-Trp-Asp-Arg- | | | | |
| | | Phe-Gly | | | | |
| CTX- | Ac | Nle-D-Ala-NMe- | 73 | OH | 200 nM | 1884 |
| 1186 | | His-Phe-Arg-D-Trp- | | | | |
| | | Asp-Arg-Phe-Gly | | | | |
| CTX- | Ac | Nle-D-Ala-NMe- | 74 | OH | 3,540 nM | 1 |
| 1189 | | Lys-Phe-Arg-D-Trp- | | | | |
| | | Asp-Arg-Phe-Gly | | | | |
| CTX- | Ac | Nle-D-Ala-NMe- | 75 | OH | 3,917 nM | |
| 1190 | | Ala-Phe-Arg-D-Trp- | | | | |
| | | Asp-Arg-Phe-Gly | | | | |
| CTX- | Ac | Nle-D-Abu-His- | 76 | OH | 3.9 nM | 470 |
| 1191 | | Phe(4-I)-Arg-D-Trp- | | | | |
| | | Asp-Arg-Phe-Gly | | | | |
| CTX- | Ac | Tyr-Val-Glu-D-Abu- | 77 | OH | 34.3 nM | 49 |
| 1194 | | His-Phe(4-I)-Arg-D- | | | | |
| | | Trp-Asp-Arg-Phe- | | | | |
| | | Gly | | | | |
| CTX- | Ac | Tyr-Val-Glu-Aib- | 78 | OH | 183.7 nM | 51 |
| 1195 | | His-Phe(4-I)-Arg-D- | | | | |
| | | Trp-Asp-Arg-Phe- | | | | |
| | | Gly | | | | |
| CTX- | Ac | Nle-D-Ala-Arg- | 79 | OH | 13.5 nM | 250 |
| 1197 | | Phe(4-I)-Arg-D-Trp- | | | | |
| | | Asp-Arg-Phe-Gly | | | | |
| CTX- | Ac | Nle-D-Ala-His- | 80 | OH | 2,800 nM | 0.01 |
| 1501 | | Phe(4-I)-D-Arg-Trp- | | | | |
| | | Asp-Arg-Phe-Gly | | | | |
| CTX- | Ac | Nle-D-Ala-His- | 81 | OH | 6.7 nM | 184 |
| 1502 | | Phe(4-I)-Arg-D- | | | | |
| | | Nal(2′)-Asp-Arg- | | | | |
| | | Phe-Gly | | | | |
| CTX- | Ac | Nle-D-Ala-His- | 82 | OH | 123.6 nM | 214 |
| 1503 | | Phe(4-I)-Arg-D-Phe- | | | | |
| | | Asp-Arg-Phe-Gly | | | | |
| CTX- | Ac | Nle-D-Ala-His- | 83 | OH | TBD | 1 |
| 1504 | | Phe(4-I)-Arg-D-Tic- | | | | |
| | | Asp-Arg-Phe-Gly | | | | |
| CTX- | Ac | Nle-D-Ala-Pro- | 84 | OH | TBD | 1 |
| 1506 | | Phe(4-I)-Arg-D-Phe- | | | | |
| | | Asp-Arg-Phe-Gly | | | | |
| CTX- | Ac | Nle-D-Ala-His-Phe- | 85 | OH | 180.7 nM | 23 |
| 1510 | | Arg-aMe-D-Trp- | | | | |
| | | Asp-Arg-Phe-Gly | | | | |
| CTX- | Ac | Nle-D-Ala-His- | 86 | OH | 13.1 nM | 804 |
| 1512 | | Nal(2')-Arg-D-Trp- | | | | |
| | | Asp-Arg-Phe-Gly | | | | |
| CTX- | Ac | Nle-D-Ala-His- | 87 | OH | 1.9 nM | 1,559 |
| 1518 | | Phe(4-F)-Arg-D- | | | | |
| | | Trp-Asp-Arg-Phe- | | | | |
| | | Gly | | | | |
| CTX- | Ac | Nle-D-Ala-His-D- | 88 | OH | 5.2 nM | 54 |
| 1520 | | Bip-Arg-D-Trp-Asp- | | | | |
| | | Arg-Phe-Gly | | | | |
| CTX- | Ac | Nle-D-Ala-His-Phe- | 89 | OH | 41.7 nM | 5,321 |
| 1523 | | Lys-D-Trp-Asp-Arg- | | | | |
| | | Phe-Gly | | | | |
| CTX- | Ac | Nle-D-Ala-His-Phe- | 90 | OH | 1.9 nM | 182 |
| 1528 | | Arg-D-Trp-Ala-Arg- | | | | |
| | | Phe-Gly | | | | |
| CTX- | Ac | Nle-D-Ala-His-Phe- | 91 | OH | 4.8 nM | 82 |
| 1529 | | Arg-D-Trp-Phe-Arg- | | | | |
| | | Phe-Gly | | | | |
| CTX- | Ac | Nle-D-Ala-His-Phe- | 92 | OH | 289 nM | 15 |
| 1530 | | Arg-D-Trp-D-Phe- | | | | |
| | | Arg-Phe-Gly | | | | |
| CTX- | Ac | Nle-D-Ala-His-Phe- | 93 | OH | 356.5 nM | 8 |
| 1531 | | Arg-D-Trp-D-Phe- | | | | |
| | | Arg-Phe-Gly | | | | |
| CTX- | Ac | Nle-D-Ala-His-Phe- | 94 | OH | 10.3 nM | 184 |
| 1532 | | Arg-D-Trp-Nle-Arg- | | | | |
| | | Phe-Gly | | | | |
| CTX- | Ac | Nle-D-Ala-His-Phe- | 95 | OH | 14.2 nM | 314 |
| 1533 | | Arg-D-Trp-Lys-Arg- | | | | |
| | | Phe-Gly | | | | |
| CTX- | Ac | Nle-D-Ala-His-Phe- | 96 | OH | 38.8 nM | 186 |
| 1534 | | Arg-D-Trp-Asp-Ala- | | | | |
| | | Phe-Gly | | | | |
| CTX- | Ac | Nle-D-Ala-His-Phe- | 97 | OH | 6 nM | 1,589 |
| 1535 | | Arg-D-Trp-Asp-Lys- | | | | |
| | | Phe-Gly | | | | |
| CTX- | Ac | Nle-D-Ala-His-Phe- | 98 | OH | 73 nM | 72 |
| 1536 | | Arg-D-Trp-Asp-Arg- | | | | |
| | | Pro-Gly | | | | |
| CTX- | Ac | Nle-D-Ala-His-Phe- | 99 | OH | 25.5 nM | 278 |
| 1537 | | Arg-D-Trp-Asp-Arg- | | | | |
| | | Ala-Gly | | | | |
| CTX- | Ac | Nle-D-Ala-His-Phe- | 100 | OH | 7.4 nM | 1,276 |
| 1538 | | Arg-D-Trp-Asp-Arg- | | | | |
| | | Phe-Val | | | | |
| CTX- | Ac | Nle-D-Ala-His-Phe- | 101 | NH2 | 47.1 nM | 16 |
| 1539 | | Arg-D-Trp | | | | |
| CTX- | Ac | Nle-D-Ala-His-Phe- | 102 | NH2 | 25.1 nM | 11 |
| 1540 | | Arg-D-Trp-Gly | | | | |
| CTX- | Ac | Nle-D-Ala-His-Phe- | 103 | NH2 | 65.2 nM | 9 |
| 1541 | | Arg-D-Trp-Phe | | | | |
| CTX- | Ac | Nle-D-Ala-His-Phe- | 104 | NH2 | 64.6 nM | 15 |
| 1542 | | Arg-D-Trp-D-Phe | | | | |
| CTX- | Ac | Nle-D-Ala-His- | 105 | NH2 | 3.1 nM | 202 |
| 1543 | | Phe(4-I)-Arg-D-Trp- | | | | |
| | | Gly | | | | |
| CTX- | Ac | Nle-Aib-His-Phe | 106 | NH2 | 51.6 nM | 14 |
| 1545 | | (4-I)-Arg-D-Trp-Gly | | | | |
| CTX- | H | Val-Met-Gly-His- | 107 | OH | 1.7 nM | 51 |
| 1546 | | Phe-Arg-D-Trp-Asp- | | | | |
| | | Arg-Phe-Gly | | | | |
| CTX- | H | Met-Gly-His-Phe- | 108 | OH | 1.5 nM | 54 |
| 1547 | | Arg-D-Trp-Asp-Arg- | | | | |
| | | Phe-Gly | | | | |
| CTX- | H | Tyr-Val-Met-Gly- | 109 | OH | 3.8 nM | 267 |
| 1548 | | Arg-Phe-Arg-D-Trp- | | | | |
| | | Asp-Arg-Phe-Gly | | | | |
| CTX- | H | Tyr-Val-Nle-Gly- | 110 | OH | 2.4 nM | 102 |
| 1549 | | Arg-Phe-Arg-D-Trp- | | | | |
| | | Asp-Arg-Phe-Gly | | | | |

### Example 2

### Plasma Stability (A)

Pooled mouse plasma was prepared and stored at −80° C. prior to use. 396 μL mouse plasma was incubated at 37° C. for 5 minutes in 1.5 mL microcentrifuge tubes. 4 μL of 100 μM test or control compound/peptide was added to each tube and incubated for 0.5, 15, 30, 60, 120, or 240 minutes. An aliquot of 40 μL of each reaction was stopped by the addition of 4 volume of cold acetonitrile containing 200 ng/mL of Setmelanotide as an internal standard (IS). The incubation solution was centrifuged at 3500 rpm for 10 minutes to precipitate protein. The supernatant was used for LC/MS/MS analysis. The natural log peak area ratio (compound peak 10 area/internal standard peak area) was plotted against time and the gradient of the line determined.

### LC-MS/MS Method:

### Chromatographic Conditions:

| Column: | Agilent Poroshell 120 SB- | |
|---|---|---|
| | C18, 2.7 μm | |
| Mobile Phase A: | 0.1% formic acid in purified | |
| | deionized water | |
| Mobile Phase B: | 0.1% formic acid in | |
| | acetonitrile | |
| Flow Rate: | 0.3 | mL/min |
| Injection | 5 | μL |
| Volume: | | |
| Run Time: | 5.5 | min |

| Time | % A | % B |
|---|---|---|
| 0.01 | 70 | 30 |
| 0.80 | 70 | 30 |
| 1.00 | 5 | 95 |
| 3.00 | 5 | 95 |
| 3.10 | 99 | 1 |
| 5.10 | 99 | 1 |

### MS/MS Conditions in 4500

### MRM-Transitions:

| | Q1 Mass | Q3 Mass | | | | |
|---|---|---|---|---|---|---|
| Compounds | (M + H⁺, m/z) | (m/z) | DP | EP | CE | CXP |
| Setmelanotide | 373.2 | 476.6 | 94.0 | 3.10 | 20.6 | 6.00 |
| (CTX-1103) | 523.0 | 653.0 | 92.8 | 6.50 | 22.3 | 7.20 |
| (CTX-1134) | 528.0 | 659.9 | 102.2 | 6.10 | 21.7 | 13.00 |
| (CTX-1108) | 402.8 | 472.3 | 120.0 | 4.50 | 11.2 | 5.40 |
| (CTX-1121) | 483.0 | 652.9 | 108.6 | 7.00 | 16.1 | 8.00 |
| (CTX-1125) | 523.4 | 235.2 | 87.2 | 6.80 | 23.3 | 7.20 |
| (CTX-1141) | 407.6 | 150.0 | 111.0 | 7.90 | 21.3 | 18.00 |
| (CTX-1126) | 523.3 | 652.8 | 111.6 | 6.60 | 19.2 | 13.00 |
| (CTX-1127) | 523.2 | 652.7 | 116.8 | 6.15 | 20.9 | 14.00 |
| (CTX-1105) | 524.0 | 653.8 | 105.0 | 6.30 | 19.8 | 8.01 |
| (CTX-2120) | 549.9 | 692.7 | 86.8 | 5.80 | 22.9 | 14.00 |
| Procainamide | 235.8 | 119.9 | 67.0 | 7.12 | 26.9 | 17.93 |
| Procaine | 236.9 | 164.1 | 105.8 | 4.05 | 14.4 | 1.39 |

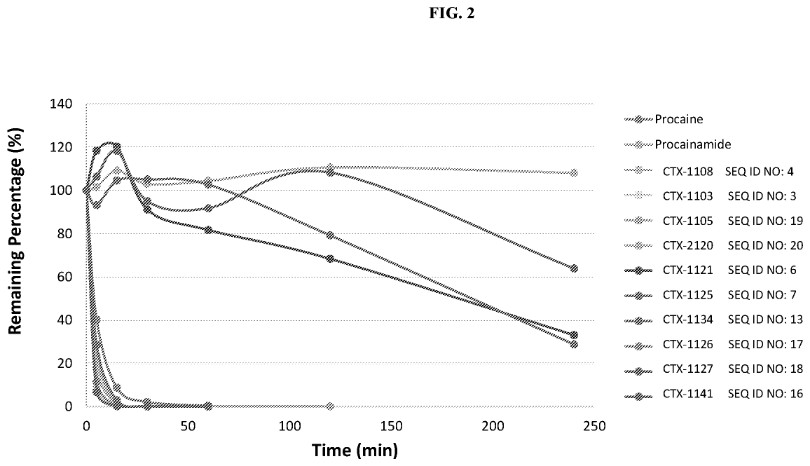

The mouse plasma stability and T1/2 of test compounds was listed in the Table 2, and plotted in FIG. 2.

TABLE 2
Mouse Plasma Stability and Half Life for 10
DTrp8-g-MSH analogs and positive control.

| Time point | | | CTX- | CTX- | CTX- | CTX- |
|---|---|---|---|---|---|---|
| (min) | Procaine | Procainamide | 1108 | 1103 | 1105 | 2120 |
| 0 | 100.00 | 100.00 | 100.00 | 100.00 | 100.00 | 100.00 |
| 5 | 40.30 | 101.53 | 7.21 | 9.30 | 23.39 | 17.85 |
| 15 | 8.80 | 109.16 | 0.33 | 0.21 | 1.51 | 0.78 |
| 30 | 2.15 | 103.05 | 0.04 | 0.03 | 0.30 | 0.13 |
| 60 | 0.44 | 104.43 | 0.01 | 0.02 | 0.27 | 0.09 |
| 120 | | 110.61 | | | 0.12 | |
| 240 | | 108.17 | | | | |
| Half time | 3.81 | >240 | 1.32 | 1.46 | 2.38 | 2.01 |
| (min) | | | | | | |

| Time point | CTX- | CTX- | CTX- | CTX- | CTX- | CTX- |
|---|---|---|---|---|---|---|
| (min) | 1121 | 1125 | 1134 | 1126 | 1127 | 1141 |
| 0 | 100.00 | 100.00 | 100.00 | 100.00 | 100.00 | 100.00 |
| 5 | 118.35 | 93.20 | 28.56 | 11.65 | 6.71 | 106.39 |
| 15 | 120.25 | 104.53 | 2.97 | 0.16 | 0.25 | 118.26 |
| 30 | 91.14 | 105.07 | 0.14 | 0.03 | 0.27 | 94.98 |
| 60 | 81.65 | 102.67 | 0.02 | | 0.04 | 91.78 |
| 120 | 68.35 | 79.20 | | | | 108.22 |
| 240 | 33.10 | 28.80 | | | | 63.93 |
| Half time | 138 | 141 | 2.77 | 1.61 | 1.28 | >240 |
| (min) | | | | | | |

Note:
Procaine and Procainamide are used as positive control for mouse plasma stability.

### Example 3

### Plasma Stability (B)

Pooled mouse plasma were prepared and stored at −80° C. prior to use. 5 μL of 100 μM test compound was added to 495 μL plasma. Aliquot of 40 μL was pipetted from the reaction solution and stopped by the addition of 160 μL cold acetonitrile containing 10 μg of CTX-1121 as an internal standard at the designated time points. The incubation solution was centrifuged at 3500 rpm for 10 minutes to precipitate protein. The supernatant was used for LC/MS/MS analysis. The natural log peak area ratio (compound peak area/internal standard peak area) was plotted against time and the gradient of the line was determined.

### LC-MS/MS Conditions

Chromatographic Conditions:
Column: Agilent Poroshell 120, 2.1×50 mm, 2.7 μm
Mobile Phase A: 0.1% formic acid in purified deionized water
Mobile Phase B: 0.1% formic acid in acetonitrile
Flow Rate: 0.3 mL/min; Injection Volume: 5 μL

| Run | % A | % B |
|---|---|---|
| Time: 5.1 min | | |
| Time(min) | | |
| 0.00 | 70 | 30 |
| 0.80 | 70 | 30 |
| 1.00 | 5 | 95 |
| 3.00 | 5 | 95 |
| 3.10 | 99 | 1 |
| 5.10 | 99 | 1 |

### MS/MS Conditions

Turbo-Ionspray™ Interface used in the positive ion-mode
MRM-transitions:

| | Q1 Mass | Q3 Mass | | | | |
|---|---|---|---|---|---|---|
| Compounds | (M + H⁺, m/z) | (m/z) | DP | EP | CE | CXP |
| CTX-1147 | 644.9 | 502.9 | 89 | 12 | 31 | 15 |
| CTX-1148 | 911.4 | 518.0 | 116 | 12 | 58 | 16 |
| CTX-1122 | 450.0 | 379.3 | 70 | 13 | 28 | 12 |
| CTX-1165 | 475.8 | 379.2 | 80 | 4 | 30 | 6 |
| CTX-1166 | 461.4 | 379.2 | 86 | 3 | 27 | 5 |

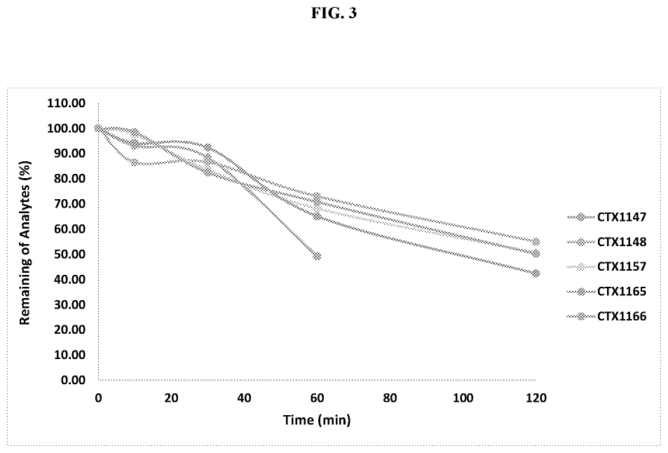

(See also FIG. 3).

### Example 4

### Chemical Synthesis

The solid-phase synthesis of several peptides (i.e., CTX-1122, CTX-1148, CTX-1151, and CTX-1161) are detailed below. All peptides were reversed-phase high performance liquid chromatography (RP-HPLC) to >95% purity and confirmed for structural integrity by liquid chromatography/mass spectrometry (LC/MS).

CTX-1122 was synthesized using standard Fmoc-based solid phase peptide synthesis on a CEM Liberty Blue synthesizer. Amino acid couplings were carried out at 90° C. for 2 min using a 5-fold excess and DIC/Oxyma activation. FMOC deprotection was achieved at 90° C. using 20% piperidine in DMF for one min. The completed peptide was deprotected and then acetylated using acetic anhydride/DIEA at 90° C. The resin-bound sequence was then cleaved and deprotected with TFA/water/thioanisole/ethylmethylsulfide/ethanedithiol (20:1:1:1:1). The peptide was precipitated into ether and then isolated by centrifugation, and the dried peptide pellet was reconstituted in a 1:1 water/acetonitrile mixture and lyophilized. The crude peptide was purified by RP-HPLC (C18, 10 μm, 25×250 mm column), using a gradient of 18-38% Buffer B in 90 minutes (Buffer A=0.1% TFA in water, Buffer B=0.1% TFA in acetonitrile). The peptide was analyzed, and pure fractions were pooled and lyophilized. Analytical LC/MS data was obtained on an analytical column (C18, 2.6 μm, 2.1×100 mm) using water-acetonitrile buffers containing 0.1% TFA.

CTX-1148 was synthesized using standard Fmoc-based solid phase peptide synthesis on a CEM Liberty Blue synthesizer up to the Gly at position 3. Double coupling of amino acids was carried out at 90° C. for 2 min using a 5-fold excess and DIC/Oxyma activation. FMOC deprotection was achieved at 90° C. using 20% piperidine in DMF for one min. Fmoc-mPEG2-OH was added manually using 2 equivalents of the amino acid and 2 equivalents of HBTU/DIEA with heat at 40° C. 3-(4-Iodophenyl)propanoic acid was added manually using 2 equivalents of the amino acid and 2 equivalents of PyAop/DIEA with heating at 40° C. The completed peptide was deprotected and then acetylated using acetic anhydride at 90° C. The resin-bound sequence was then cleaved and deprotected with TFA/water/thioanisole/ethylmethylsulfide/ethanedithiol (20:1:1:1:1). The peptide was precipitated into ether and then isolated by centrifugation. The dried peptide pellet was reconstituted in a 1:1 water/acetonitrile mixture and lyophilized. The crude peptide was purified by RP-HPLC (C18, 10 μm, 25×250 mm column), using a gradient of 25-45% Buffer B in 120 minutes (Buffer A=0.1% TFA in water, Buffer B=0.1% TFA in acetonitrile). The peptide was analyzed, and pure fractions were pooled and lyophilized. Analytical LC/MS data was obtained on an analytical column (C18, 2.6 μm, 2.1×100 mm) using water-acetonitrile buffers containing 0.1% TFA.

CTX-1151 was synthesized using standard Fmoc-based solid phase peptide synthesis on a CEM Liberty Blue synthesizer up to the Gly at position 3. Double coupling of amino acids was carried out at 90° C. for 2 min using a 5-fold excess and DIC/Oxyma activation. FMOC deprotection was achieved at 90° C. using 20% piperidine in DMF for one min. Fmoc-mPEG2-OH and Octadecanedioic acid mono-tert-butyl ester were added manually using 2 equivalents of the amino acid and 2 equivalents of HBTU/DIEA with heat at 40° C. The resin-bound sequence was then cleaved and deprotected with TFA/water/thioanisole/ethylmethylsulfide/ethanedithiol (20:1:1:1:1). The peptide was precipitated into ether and then isolated by centrifugation. The dried peptide pellet was reconstituted in a 1:1 water/acetonitrile mixture and lyophilized. The crude peptide was purified by RP-HPLC (C18, 10 μm, 25×250 mm column), using a gradient of 37-57% Buffer B in 120 minutes (Buffer A=0.1% TFA in water, Buffer B=0.1% TFA in acetonitrile). The peptide was analyzed, and pure fractions were pooled and lyophilized. Analytical LC/MS data was obtained on an analytical column (C18, 2.6 μm, 2.1×100 mm) using water-acetonitrile buffers containing 0.1% TFA.

CTX-1165 was synthesized using standard Fmoc-based solid phase peptide synthesis on a CEM Liberty Blue synthesizer. Double coupling of amino acids was carried out at 90° C. for 2 min using a 5-fold excess and DIC/Oxyma activation. FMOC deprotection was achieved at 90° C. using 20% piperidine in DMF for one min. The completed peptide was deprotected and then acetylated using acetic anhydride/DIEA at 90° C. The resin-bound sequence was then cleaved and deprotected with TFA/water/thioanisole/ethylmethylsulfide/ethanedithiol (20:1:1:1:1). The peptide was precipitated into ether and then isolated by centrifugation. The dried peptide pellet was reconstituted in a 1:1 water/acetonitrile mixture and lyophilized. The crude peptide was purified by RP-HPLC (C18, 10 μm, 25×250 mm column), using a gradient of 21-41% Buffer B in 100 minutes (Buffer A=0.1% TFA in water, Buffer B=0.1% TFA in acetonitrile). The peptide was analyzed, and pure fractions were pooled and lyophilized. Analytical LC/MS data was obtained on an analytical column (C18, 2.6 μm, 2.1×100 mm) using water-acetonitrile buffers containing 0.1% TFA.

SEQUENCES

SEQ ID NO: 1
X-AA0-AA1-AA2-AA3-AA4-AA5-AA6-AA7-AA8-AA9-AA10-
AA11-AA12-Y (SEQ ID NO: 1);
SEQ ID NO: 2
Tyr-Val-Met-Gly-His-Phe-Arg-D-Trp-Asp-Arg-Phe-Gly
SEQ ID NO: 3
Tyr-Val-Met-Gly-His-Phe-Arg-D-Trp-Asp
SEQ ID NO: 4
Gly-His-Phe-Arg-D-Trp-Asp-Arg-Phe-Gly
SEQ ID NO: 5
Gly-His-Phe-Arg-D-Trp-Asp
SEQ ID NOS: 6-110
See Table 1

### REFERENCES

The following references, some of which cited above by number, are herein incorporated by reference in their entireties.
1. Andermann, M. L. & Lowell, B. B. Toward a Wiring Diagram Understanding of Appetite Control. Neuron (2017). doi:10.1016/j.neuron.2017.06.014
2. Sternson, S. M., Nicholas Betley, J. & Cao, Z. F. H. Neural circuits and motivational processes for hunger. Current Opinion in Neurobiology (2013). doi: 10.1016/j.conb.2013.04.006
3. Sohn, J. W., Elmquist, J. K. & Williams, K. W. Neuronal circuits that regulate feeding behavior and metabolism. Trends in Neurosciences (2013). doi:10.1016/j.tins.2013.05.003
4. Mercer, A. J., Hentges, S. T., Meshul, C. K. & Low, M. J. Unraveling the central proopiomelanocortin neural circuits. Front. Neurosci. (2013). doi: 10.3389/fnins.2013.00019
5. Cone, R. D. Anatomy and regulation of the central melanocortin system. Nature Neuroscience (2005). doi:10.1038/nn1455
6. Sternson, S. M. & Atasoy, D. Agouti-related protein neuron circuits that regulate appetite. Neuroendocrinology (2014). doi: 10.1159/000369072
7. Krashes, M. J., Shah, B. P., Koda, S. & Lowell, B. B. Rapid versus delayed stimulation of feeding by the endogenously released agRP neuron mediators GABA, NPY, and AgRP. Cell Metab. (2013). doi:10.1016/j.cmet.2013.09.009
8. Zhan, C. et al. Acute and Long-Term Suppression of Feeding Behavior by POMC Neurons in the Brainstem and Hypothalamus, Respectively. J. Neurosci. (2013). doi: 10.1523/jneurosci.2742-12.2013
9. Betley, J. N., Cao, Z. F. H., Ritola, K. D. & Sternson, S. M. Parallel, redundant circuit organization for homeostatic control of feeding behavior. Cell (2013). doi: 10.1016/j.cell.2013.11.002
10. Atasoy, D., Nicholas Betley, J., Su, H. H. & Sternson, S. M. Deconstruction of a neural circuit for hunger. Nature (2012). doi:10.1038/nature11270
11. Luquet, S., Perez, F. A., Hnasko, T. S. & Palmiter, R. D. NPY/AgRP neurons are essentials for feeding in adult mice but can be ablated in neonates. Science (80-.). (2005). doi: 10.1126/science.1115524
12. Krashes, M. J. et al. Rapid, reversible activation of AgRP neurons drives feeding behavior in mice. J. Clin. Invest. (2011). doi: 10.1172/JCI46229
13. Aponte, Y., Atasoy, D. & Sternson, S. M. AGRP neurons are sufficient to orchestrate feeding behavior rapidly and without training. Nat. Neurosci. (2011). doi: 10.1038/nn.2739
14. Burnett, C. J. et al. Hunger-Driven Motivational State Competition. Neuron (2016). doi: 10.1016/j.neuron.2016.08.032
15. Padilla, S. L. et al. Agouti-related peptide neural circuits mediate adaptive behaviors in the starved state. Nat. Neurosci. (2016). doi: 10.1038/nn.4274
16. Bagnol, D. et al. Anatomy of an endogenous antagonist: relationship between Agouti-related protein and proopiomelanocortin in brain. J. Neurosci. (1999).
17. Roselli-Rehfuss, L. et al. Identification of a receptor for gamma melanotropin and other proopiomelanocortin peptides in the hypothalamus and limbic system. Proc. Natl. Acad. Sci. (2006). doi: 10.1073/pnas.90.19.8856
18. Ghamari-Langroudi, M. et al. Regulation of energy rheostasis by the melanocortin-3 receptor. Sci. Adv. (2018). doi: 10.1126/sciadv.aat0866
19. Sutton, G. M. et al. The Melanocortin-3 Receptor Is Required for Entrainment to Meal Intake. J. Neurosci. (2008). doi:10.1523/JNEUROSCI.3615-08.2008
20. Renquist, B. J. et al. Melanocortin-3 receptor regulates the normal fasting response. Proc. Natl. Acad. Sci. (2012). doi: 10.1073/pnas. 1201994109
21. Fenselau, H. et al. A rapidly acting glutamatergic ARC→PVH satiety circuit postsynaptically regulated by α-MSH. Nat. Neurosci. (2017). doi: 10.1038/nn.4442
22. Armbruster, B. N., Li, X., Pausch, M. H., Herlitze, S. & Roth, B. L. Evolving the lock to fit the key to create a family of G protein-coupled receptors potently activated by an inert ligand. Proc. Natl. Acad. Sci. (2007). doi:10.1073/pnas.0700293104
23. Alexander, G. M. et al. Remote Control of Neuronal Activity in Transgenic Mice Expressing Evolved G Protein-Coupled Receptors. Neuron (2009). doi: 10.1016/j.neuron.2009.06.014
24. Stachniak, T. J., Ghosh, A. & Sternson, S. M. Chemogenetic Synaptic Silencing of Neural Circuits Localizes a Hypothalamus→Midbrain Pathway for Feeding Behavior. Neuron (2014). doi: 10.1016/j.neuron.2014.04.008
25. Li, C. et al. AGRP neurons modulate fasting-induced anxiolytic effects. Transl. Psychiatry (2019). doi: 10.1038/s41398-019-0438-1
26. Dietrich, M. O., Zimmer, M. R., Bober, J. & Horvath, T. L. Hypothalamic Agrp neurons drive stereotypic behaviors beyond feeding. Cell (2015). doi:10.1016/j.cell.2015.02.024
27. Zelikowsky, M. et al. The Neuropeptide Tac2 Controls a Distributed Brain State Induced by Chronic Social Isolation Stress. Cell (2018). doi: 10.1016/j.cell.2018.03.037
28. Ieraci, A., Mallei, A. & Popoli, M. Social Isolation Stress Induces Anxious-Depressive-Like Behavior and Alterations of Neuroplasticity-Related Genes in Adult Male Mice. Neural Plast. (2016). doi: 10.1155/2016/6212983
29. Buynitsky, T. & Mostofsky, D. I. Restraint stress in biobehavioral research: Recent developments. Neuroscience and Biobehavioral Reviews (2009). doi: 10.1016/j.neubiorev.2009.05.004
30. Campos, A. C., Fogaça, M. V., Aguiar, D. C. & Guimarães, F. S. Animal models of anxiety disorders and stress. Rev. Bras. Psiquiatr. (2013). doi: 10.1590/1516-4446-2013-1139
31. Grieco, P., Balse, P. M., Weinberg, D., MacNeil, T. & Hruby, V. J. D-amino acid scan of y-melanocyte-stimulating hormone: Importance of Trp8 on human MC3 receptor selectivity. J. Med. Chem. (2000). doi:10.1021/jm000211e
32. Merlino, F. et al. Development of Macrocyclic Peptidomimetics Containing Constrained α,α-Dialkylated Amino Acids with Potent and Selective Activity at Human Melanocortin Receptors. J. Med. Chem. (2018). doi: 10.1021/acs.jmedchem.8b00488
33. Carotenuto, A. et al. Discovery of Novel Potent and Selective Agonists at the Melanocortin-3 Receptor. J. Med. Chem. (2015). doi:10.1021/acs.jmedchem.5b01285
34. Herpertz-dahlmann, B., Holtkamp, K. & Konrad, K. Eating disorders: Anorexia and bulimia nervosa. Handb. Clin. Neurol. (2012). doi:10.1016/B978-0-444-52002-9.00026-7
35. Smink, F. R. E., Van Hoeken, D. & Hoek, H. W. Epidemiology, course, and outcome of eating disorders. Current Opinion in Psychiatry (2013). doi: 10.1097/YCO.0b013e328365a24f
36. Jerlhag, E. et al. Ghrelin stimulates locomotor activity and accumbal dopamine-overflow via central cholinergic systems in mice: Implications for its involvement in brain reward. Addict. Biol. (2006). doi: 10.1111/j.1369-1600.2006.00002.x
37. Adermark, L. et al. Ghrelin administration into tegmental areas stimulates locomotor activity and increases extracellular concentration of dopamine in the nucleus accumbens. Addict. Biol. (2007). doi: 10.1111/j.1369-1600.2006.00041.x
38. Marks, D. L., Hruby, V., Brookhart, G. & Cone, R. D. The regulation of food intake by selective stimulation of the type 3 melanocortin receptor (MC3R). Peptides (2006). doi: 10.1016/j.peptides.2005.01.025
39. Lee, M. et al. Effects of selective modulation of the central melanocortin-3-receptor on food intake and hypothalamic POMC expression. Peptides (2008). doi: 10.1016/j.peptides.2007.11.005
40. Lippert, R. N., Ellacott, K. L. J. & Cone, R. D. Gender-specific roles for the melanocortin-3 receptor in the regulation of the mesolimbic dopamine system in mice. Endocrinology (2014). doi:10.1210/en.2013-2049
41. Pandit, R. et al. Melanocortin 3 receptor signaling in midbrain dopamine neurons increases the motivation for food reward. Neuropsychopharmacology (2016). doi: 10.1038/npp.2016.19
42. Mavrikaki, M. et al. Melanocortin-3 receptors in the limbic system mediate feeding-related motivational responses during weight loss. Mol. Metab. (2016). doi: 10.1016/j.molmet.2016.05.002
43. Pei, H. et al. Lateral Hypothalamic Mc3R-Expressing Neurons Modulate Locomotor Activity, Energy Expenditure, and Adiposity in Male Mice. Endocrinology 160, 343-358 (2018).
44. Sternson, S. M. & Eiselt, A.-K. Three Pillars for the Neural Control of Appetite. Annu. Rev. Physiol. (2016). doi: 10.1146/annurev-physiol-021115-104948
45. Rossi, M. A. & Stuber, G. D. Overlapping Brain Circuits for Homeostatic and Hedonic Feeding. Cell Metabolism (2018). doi: 10.1016/j.cmet.2017.09.021
46. Sweeney, P. & Yang, Y. Neural Circuit Mechanisms Underlying Emotional Regulation of Homeostatic Feeding. Trends in Endocrinology and Metabolism (2017). doi: 10.1016/j.tem.2017.02.006
47. Hay, P. J., Touyz, S. & Sud, R. Treatment for severe and enduring anorexia nervosa: A review: Australian and New Zealand Journal of Psychiatry (2012). doi: 10.1177/0004867412450469
48. Bulik, C. M. et al. Prevalence, heritability, and prospective risk factors for anorexia nervosa. Arch. Gen. Psychiatry (2006). doi: 10.1001/archpsyc.63.3.305
49. Keski-Rahkonen, A. et al. Epidemiology and course of anorexia nervosa in the community. Am. J. Psychiatry (2007). doi: 10.1176/appi.ajp.2007.06081388
50. Marks, D. L., Butler, A. A., Turner, R., Brookhart, G. & Cone, R. D. Differential role of melanocortin receptor subtypes in cachexia. Endocrinology (2003). doi: 10.1210/en.2002-221099
51. Fazeli, P. K. et al. Treatment with a ghrelin agonist in outpatient women with anorexia nervosa: A randomized clinical trial. J. Clin. Psychiatry (2018). doi: 10.4088/JCP.17m11585
52. Fazeli, P. et al. Short-term treatment with a ghrelin agonist significantly improves gastric emptying in anorexia nervosa. Endocrine reviews. Conference: 98th annual meeting and expo of the endocrine society, ENDO 2016. United states. Conference start: 20160401. Conference end: 20160404 (2016). doi: 10.1210/endo-meetings. 2016.OABA.2.SUN-606
53. Carlini, V. P. et al. Ghrelin increases anxiety-like behavior and memory retention in rats. Biochem. Biophys. Res. Commun. (2002).
54. Carvajal, P., Carlini, V. P., Schioth, H. B., de Barioglio, S. R. & Salvatierra, N. A. Central ghrelin increases anxiety in the Open Field test and impairs retention memory in a passive avoidance task in neonatal chicks. Neurobiol. Learn. Mem. (2009). doi: 10.1016/j.nlm.2008.12.008
55. Sweeney, P. & Yang, Y. An excitatory ventral hippocampus to lateral septum circuit that suppresses feeding. Nat. Commun. (2015). doi: 10.1038/ncomms10188
56. Sweeney, P. & Yang, Y. An Inhibitory Septum to Lateral Hypothalamus Circuit That Suppresses Feeding. J. Neurosci. (2016). doi: 10.1523/jneurosci.2042-16.2016
57. Sweeney, P., Li, C. & Yang, Y. Appetite suppressive role of medial septal glutamatergic neurons. Proc. Natl. Acad. Sci. (2017). doi: 10.1073/pnas. 1707228114
58. Singh, A., Dirain, M., Witek, R., Rocca J. R., Edison, A. S., and Haskell-Luevano, C. Structure-Activity Relationships of Peptides Incorporating a Bioactive Reverse-Turn Heterocycle at the Melanocortin Receptors: Identification of a 5800-fold Mouse Melanocortin-3 Receptor (mMC3R) Selective Antagonist/Partial Agonist versus the Mouse Melanocortin-4 Receptor (mMC4R). J. Med. Chem. 56, 2747-2763, 2013.
59. L. B. Knudsen et al. and H. Agerso. Potent derivatives of glucagon-like-peptide 1 with pharmacokinetic properties suitable for once daily administration. J. Med. Chem., 43, 1664-1669, 2000.
60. J. Lau et al. and T. Kruse. Discovery of the once-weekly glucagon-like peptide-1 (GLP-1) analogue semaglutide. J. Med. Chem., 58, 7370-7380, 2015.
61. C. E. Dumelin et al. and D. Neri. A portable albumin binder from a DNA-encoded chemical library. Angew. Chm. Int. Ed., 47, 3196-3201, 2008.
62. B. Y. H. Lam et al. and S. O'Rahilly. MC3R links nutritional state to childhood growth and the timing of puberty. Nature 599, 436-441, 2021.

1. A composition comprising a peptide having 4 or fewer substitutions relative to the sequence:
(SEQ ID NO: 1)
X-AA0-AA1-AA2-AA3-AA4-AA5-AA6-AA7-AA8-AA9-AA10-
AA11-AA12-Y;
wherein X is a N-terminal cap moiety linked to the most N-terminal amino acid of the peptide and is acetyl, C18 diacid-Glu-PEG-Gly, Aryl(4-I)-PEG-Gly, or absent;
wherein AA0 is absent or Gly;
wherein AA1 is Tyr, D-Tyr, NMe-Tyr, or absent;
wherein AA2 is Val, Gly, Ala, Aib, or absent;
wherein AA3 is Met, Nle, Glu, Ser, Asp, homoGlu, or Thr;
wherein AA4 is Gly, D-Ala, D-Val, D-Nle, NMe-D-Ala, D-Pro, Ala, Aib, D-Abu, D-Phe, Glu, or absent;
wherein AA5 is His, Pro, Gln, Cit, NMe-His, NMe-Arg, NMe-Lys, NMe-Ala;
wherein AA6 is Phe, D-Phe, Nal(2′), Trp, D-Trp, Tic, Hph, Bip, D-Bip, aMe-Phe, D-Phe(4-NH—Ac), Phe(4-F), Phe(4tBu), Phe(4-Br), Phe(4-I), Phe(4-Cl), D-Phe(4-Br), D-Phe(4-I), NMe-Phe, or D-Phe(4-Cl);
wherein AA7 is Arg, D-Arg, Lys, Orn, Cit, or Nle;
wherein AA8 is aMe-D-Trp, D-Trp, Trp, D-Nal(2′), D-Phe, D-Tic, Tic, D-Phe, or D-Ala;
wherein AA9 is Asp, Ala, Phe, D-Phe, Nle, Lys, Gly, Orn, or absent;
wherein AA10 is Arg, D-Arg, Lys, Ala, or absent;
wherein AA11 is Phe, D-Phe, Pro, Gly, Ala, or absent;
wherein AA12 is Gly, Lys, Val, or absent;
wherein Y is a C-terminal cap linked to the most C-terminal amino acid of the peptide and is NH₂ or absent;
wherein AA2 is present if AA1 is present;
wherein AA2 and AA1 are present in AA0 is present;
wherein AA10 and AA11 are present if AA12 is present;
wherein AA10 is present if AA11 is present; and
wherein the peptide does not consist of Tyr-Val-Met-Gly-His-Phe-Arg-D-Trp-Asp-Arg-Phe-Gly (SEQ ID NO: 2).

2. The composition of claim 1, wherein the peptide comprises 100% sequence similarity to the sequence:
(SEQ ID NO: 1)
X-AA0-AA1-AA2-AA3-AA4-AA5-AA6-AA7-AA8-AA9-AA10-
AA11-AA12-Y;
wherein X is a N-terminal cap moiety linked to the most N-terminal amino acid of the peptide and is acetyl, C18 diacid-Glu-PEG-Gly, Aryl(4-I)-PEG-Gly, or absent;
wherein AA0 is absent or Gly;
wherein AA1 is Tyr, D-Tyr, NMe-Tyr, or absent;
wherein AA2 is Val, Gly, Ala, Aib, or absent;
wherein AA3 is Met, Nle, Glu, Ser, Asp, homoGlu, or Thr;
wherein AA4 is Gly, D-Ala, D-Val, D-Nle, NMe-D-Ala, D-Pro, Ala, Aib, D-Abu, D-Phe, Glu, or absent;
wherein AA5 is His, Pro, Gln, Cit, NMe-His, NMe-Arg, NMe-Lys, NMe-Ala;
wherein AA6 is Phe, D-Phe, Nal(2), Trp, D-Trp, Tic, Hph, Bip, D-Bip, aMe-Phe, D-Phe(4-NH—Ac), Phe(4-F), Phe(4tBu), Phe(4-Br), Phe(4-I), Phe(4-Cl), D-Phe(4-Br), D-Phe(4-I), NMe-Phe, or D-Phe(4-Cl);
wherein AA7 is Arg, D-Arg, Lys, Orn, Cit, or Nle;
wherein AA8 is aMe-D-Trp, D-Trp, Trp, D-Nal(2′), D-Phe, D-Tic, Tic, D-Phe, or D-Ala;
wherein AA9 is Asp, Ala, Phe, D-Phe, Nle, Lys, Gly, Orn, or absent;
wherein AA10 is Arg, D-Arg, Lys, Ala, or absent;
wherein AA11 is Phe, D-Phe, Pro, Gly, Ala, or absent;
wherein AA12 is Gly, Lys, Val, or absent;
wherein Y is a C-terminal cap linked to the most C-terminal amino acid of the peptide and is NH₂ or absent;
wherein AA2 is present if AA1 is present;
wherein AA2 and AA1 are present in AA0 is present;
wherein AA10 and AA11 are present if AA12 is present;
wherein AA10 is present if AA11 is present; and
wherein the peptide does not consist of Tyr-Val-Met-Gly-His-Phe-Arg-D-Trp-Asp-Arg-Phe-Gly (SEQ ID NO: 2).

3. The composition of claim 1, wherein the peptide comprises the sequence:
(SEQ ID NO: 1)
X-AA0-AA1-AA2-AA3-AA4-AA5-AA6-AA7-AA8-AA9-AA10-
AA11-AA12-Y;
wherein X is a N-terminal cap moiety linked to the most N-terminal amino acid of the peptide and is acetyl, C18 diacid-Glu-PEG-Gly, Aryl(4-I)-PEG-Gly, or absent;
wherein AA0 is absent or Gly;
wherein AA1 is Tyr, D-Tyr, NMe-Tyr, or absent;
wherein AA2 is Val, Gly, Ala, Aib, or absent;
wherein AA3 is Met, Nle, Glu, Ser, Asp, homoGlu, or Thr;
wherein AA4 is Gly, D-Ala, D-Val, D-Nle, NMe-D-Ala, D-Pro, Ala, Aib, D-Abu, D-Phe, Glu, or absent;
wherein AA5 is His, Pro, Gln, Cit, NMe-His, NMe-Arg, NMe-Lys, NMe-Ala;
wherein AA6 is Phe, D-Phe, Nal(2′), Trp, D-Trp, Tic, Hph, Bip, D-Bip, aMe-Phe, D-Phe(4-NH—Ac), Phe(4-F), Phe(4tBu), Phe(4-Br), Phe(4-I), Phe(4-Cl), D-Phe(4-Br), D-Phe(4-I), NMe-Phe, or D-Phe(4-Cl);
wherein AA7 is Arg, D-Arg, Lys, Orn, Cit, or Nle;
wherein AA8 is aMe-D-Trp, D-Trp, Trp, D-Nal(2′), D-Phe, D-Tic, Tic, D-Phe, or D-Ala;
wherein AA9 is Asp, Ala, Phe, D-Phe, Nle, Lys, DGly, Om, or absent;
wherein AA10 is Arg, D-Arg, Lys, Ala, or absent;
wherein AA11 is Phe, D-Phe, Pro, Gly, Ala, or absent;
wherein AA12 is Gly, Lys, Val, or absent;
wherein Y is a C-terminal cap linked to the most C-terminal amino acid of the peptide and is NH₂ or absent;
wherein AA2 is present if AA1 is present;
wherein AA2 and AA1 are present in AA0 is present;
wherein AA10 and AA11 are present if AA12 is present;
wherein AA10 is present if AA11 is present; and
wherein the peptide does not consist of Tyr-Val-Met-Gly-His-Phe-Arg-D-Trp-Asp-Arg-Phe-Gly (SEQ ID NO: 2).

4. A composition comprising a peptide having 1-4 substitutions or terminal deletions relative to the amino acid sequence Tyr-Val-Met-Gly-His-Phe-Arg-D-Trp-Asp-Arg-Phe-Gly (SEQ ID NO: 2).

5. A composition comprising a peptide having 1-3 substitutions relative to the amino acid sequence Tyr-Val-Met-Gly-His-Phe-Arg-D-Trp-Asp (SEQ ID NO: 3).

6. A composition comprising a peptide having 1-3 substitutions relative to the amino acid sequence Gly-His-Phe-Arg-D-Trp-Asp-Arg-Phe-Gly (SEQ ID NO: 4).

7. A composition comprising a peptide having 1 or 2 or fewer substitutions relative to the amino acid sequence Gly-His-Phe-Arg-D-Trp-Asp (SEQ ID NO: 5).

8. The composition of one of claims 1-7, wherein the peptide is selected from one or SEQ ID NOS: 2-110.

9. The composition of claim 8, wherein the peptide is selected from one or SEQ ID NOS: 6-110.

10. The composition of one of claims 1-7, wherein the peptide comprises one or more non-proteinogenic amino acids or amino acid analogs.

11. The composition of one of claims 1-10, wherein the peptide is selective for melanocortin 3 receptor (MC3R) over melanocortin 4 receptor (MC4R).

12. The composition of one of claims 1-10, wherein the peptide is a melanocortin 3 receptor (MC3R) agonist.

13. A method of treating an eating disorder comprising administering a composition of one of claims 1-12 to a subject suffering from the eating disorder.

14. The method of claim 13, wherein the eating disorder is characterized by under eating.

15. The method of claim 13, wherein the eating disorder is characterized by one or more emotional/mental symptoms.

16. The method of claim 13, wherein the eating disorder is characterized by anxiety and/or depression.

17. The method of claim 13, wherein the eating disorder is anorexia nervosa.

18. The method of claim 13, wherein the composition is co-administered with nutritional therapy, psychotherapy, nasogastric feeding, antidepressant agents, and/or antipsychotic agents.

19. A method of treating an emotional/mental disorder comprising administering a composition of one of claims 1-12 to a subject suffering from the emotional/mental disorder.

20. The method of claim 19, wherein the emotional/mental disorder is characterized by anxiety and/or depression.

21. The method of claim 19, wherein the composition is co-administered with psychotherapy, antianxiety agents, mood stabilizers, stimulants, antidepressant agents, and/or antipsychotic agents.

22. The method of one of claims 13-21, wherein the administration is repeated on a recurring basis for a period of at least 1 week.

23. The method of claim 22, wherein the administration is repeated on a daily basis.

24. The method of claim 22, wherein the administration is repeated on a recurring basis for a period of at least 1 month.

25. The method of claim 24, wherein the administration is repeated on a recurring basis for a period of at least 1 year.

26. Use of a composition of one of claims 1-12 in the treatment or prevention of an eating disorder and/or emotional/mental disorder.

27. Use of a composition of one of claims 1-12 as a medicament.

28. A composition of one of claims 1-12 for use in the manufacture of a medicament.
